(19) United States
(12) Patent Application Publication
(10) Pub. No.: US 2025/0369055 A1
(43) Pub. Date: Dec. 4, 2025

(54) Kits and Methods for Testing for Lung Cancer Risks, and Diagnosis of Disease and Disease Risk

(71) Applicant: The University of Toledo, Toledo, OH (US)

(72) Inventors: James C. Willey, Toledo, OH (US); Daniel J. Craig, Toledo, OH (US); Thomas M. Blomquist, Toledo, OH (US); Erin L. Crawford, Toledo, OH (US); Ji-Youn Yeo, Toledo, OH (US)

(73) Assignee: The University of Toledo, Toledo, OH (US)

(21) Appl. No.: 19/292,174

(22) Filed: Aug. 6, 2025

Related U.S. Application Data
(62) Division of application No. 17/640,610, filed on Mar. 4, 2022.
(60) Provisional application No. 62/897,343, filed on Sep. 8, 2019.

(51) Int. Cl. C12Q 1/6886; C12Q 1/6806

(52) U.S. Cl. CPC C12Q 1/6886; C12Q 1/6806; C12Q 2600/118; C12Q 2600/16

(57) ABSTRACT

Kits and methods for diagnosing risk of developing lung cancers and uses thereof are described.

### CROSS-REFERENCE TO RELATED APPLICATIONS

This is a divisional application U.S. application Ser. No. 17/640,610 filed on Mar. 4, 2022, which is a national stage application filed under 35 USC § 371 of international application PCT/US2020/049629 filed Sep. 8, 2020, which claims the benefit of U.S. Provisional Application No. 62/897,343 filed Sep. 8, 2019, the entire disclosures of which are expressly incorporated herein by reference.

### STATEMENT REGARDING FEDERALLY SPONSORED RESEARCH

This invention was made with government support under Grant Number CA086368 awarded by the National Institutes of Health and Early Detection Research Network Sub-Award 0000921356 awarded by the National Cancer Institute. The government has certain rights in the invention.

### FIELD OF THE INVENTION

The present invention relates to kits and methods for testing lung cancer risks.

### BACKGROUND

Lung cancer is the leading cause of cancer-related death in men and women, and cigarette smoking is the most significant preventable risk factor. Despite widespread smoking cessation initiatives, due to past and continued cigarette use, as well as the lack of effective treatment for advanced disease, lung cancer will continue to be the deadliest cancer for decades to come.

The primary strategies to reduce lung cancer death are prevention through reduction in exposure to tobacco products and screening of high-risk subjects by annual low-dose CT (LDCT) scan to diagnose lung cancer when it is in early stage and curable. Annual LDCT screening significantly reduces lung cancer mortality. However, there is large inter-individual variation in lung cancer risk among those currently recommended for screening according to demographic criteria. Overall, lung cancer incidence is low (i.e., <10%) among those who currently meet screening criteria, and this is associated with low positive predictive value and specificity.

However, one challenge is that cancers contain many unique population sub-clones. Mutations providing resistance are selected for survival when sensitive clones are killed. The current strategy is to re-sample when resistance develops and identify new dominant clone. However, identifying resistant sub-clones and potential drivers is dependent on assay level of detail. Also, traditional NGS methods create signal artifacts due to multiple sources of imprecision making identification of mutations with variant allele fraction (VAF)<2.5% difficult.

In addition, some non-limiting examples of sources of imprecision in clinical NGS include technical errors due to library preparation (amplicon and hybrid capture) that involves PCR amplification, which introduces errors at a rate that corresponds to polymerase infidelity (˜10⁻⁴); and, sequencing where each Next Generation Sequencing (NGS) platform has a nucleotide substitution error rate associated with it that limits its ability to accurately sequence a strand of DNA.

Other sources of imprecision in clinical NGS include variation in sample quantity resulting in stochastic sampling errors. Diagnostic samples may be limiting because, for example, fine-needle aspirate (FNA) yields little material beyond that necessary for cytologic analysis; and/or core biopsies yield little beyond that necessary for histologic analysis. In addition, circulating tumor DNA (ctDNA) is highly variable and dependent on disease progression such that measurable genome copies is often limiting in a plasma sample.

Other sources of imprecision in clinical NGS include sample quality errors where DNA may be damaged during processing and result in a higher rate of technical error not representative of true biological variation. For example, sources of DNA damage occur during processing including the Formalin-Fixed Paraffin-Embedded (FFPE) method of preservation of cell tissues, and during DNA extraction and sequencing protocols. Much evidence indicates FFPE damage is systematic and time-dependent.

Therefore, both standardization and quality control is needed to provide inter-lab harmonization for low-frequency variant calling.

For example, in a recent study, targeted NGS capable of measuring mutations with variant allele frequency (VAF)>1.0% was used to assess driver gene somatic mutations in lung cancer tissue and adjacent matched normal tissue from a group of subjects. A large number of mutations known to be drivers for lung cancer were identified in non-cancer lung tissues in close proximity to each cancer. As such, measurement of mutations with VAF>1% may support development of biomarkers for early diagnosis and/or genetic characterization of a prevalent lung cancer. However, the clone prevalence diminished proportional to the distance from the cancer site, with very few mutants in the normal airway of the lung not affected by the cancer or in nasal epithelium. As such, this approach did not support development of a non-invasive test for future incidental lung cancer risk. (Kadara H, Sivakumar S, Jakubek Y, San Lucas FA, Lang W, McDowell T, et al., Mutations in Normal Airway Epithelium Elucidate Spatiotemporal Resolution of Lung Cancer, Am J Respir Crit Care Med., 2019).

Thus, there is need for methods and kits that will enable NGS measurement a combination of test features that are highly associated with lung cancer risk, and also better control for quantitative and qualitative technical errors associated with NGS. Meeting these needs will allow more accurate stratification of individuals according to lung cancer risk and thereby reduce cost and harms related to LCDT screening.

### SUMMARY OF THE INVENTION

In a first aspect, described herein are lung cancer risk test kits that include reagents for measurement of multiple low VAF (defined as VAF<1%) mutants in a set of lung cancer driver genes; and, instructions therefor.

In certain embodiments, the kit comprises reagents for measurement of expression and/or somatic mutations in multiple genes in normal airway epithelial cells by next generation sequencing, the kit including: PCR primers for each target gene, synthetic internal standard for each target gene, and reagents to prepare PCR products as a library for next generation sequencing.

In certain embodiments, the kit comprises reagents for measurement of expression and/or somatic mutations in multiple genes in normal airway epithelial cells by next generation sequencing, the kit including: DNA capture probes for each target gene, synthetic internal standard for each target gene, and reagents to prepare bait-capture products as a library for next generation sequencing.

In certain embodiments, VAF<0.01%.

In certain embodiments, the VAF is about 5×10−4 (0.05%).

In certain embodiments, inclusion of the internal standards reliably measures mutations at a variant frequency as low as 0.05%, and 5% without the inclusion of the internal standards.

In certain embodiments, inclusion of the internal standards reliably measures mutations at a variant frequency as low as 0.05%.

In certain embodiments, the kit or method enables measurement of VAF as low as 0.05% without any qualifications (i.e., 5% without inclusion).

In certain embodiments, synthetic internal standards are included.

In certain embodiments, the lung cancer risk associated driver genes comprise one or more of: TP53, PIK3CA, BRAF, KRAS, NRAS, NOTCHI, EGFR, and ERBB2.

In certain embodiments, the lung cancer driver risk associated genes comprise one or more of: CDKN1A, E2F1, ERCC1, ERCC4, ERCC5, GPX1, GSTP1, KEAP1, RB1, TP63, and XRCC1.

In certain embodiments, the analytes are measured in RNA or DNA from airway epithelial cells.

In certain embodiments, the analytes are measured in non-invasively obtained specimens, including exhaled breath condensate and/or airway epithelial cells obtained by nasal brushings.

In certain embodiments, the each kit or method provides reagents and instructions necessary for measurement of multiple analytes comprised by one or more lung cancer risk tests.

In certain embodiments, each kit or method is used to measure each analyte comprised by each test in multiple patient specimens.

In another aspect, described herein are methods of diagnosing whether a subject is at risk of developing lung cancer. In one embodiment, the method comprises:
obtaining a biological sample from the subject;
measuring the levels of set of lung cancer driver genes in the biological sample using any one of the kits of any one of the claims herein so as to obtain physical data to determine whether the levels in the biological sample is higher than the levels in a control; comparing the levels in the biological sample with the levels in the control;
distinguishing between true mutations and artifacts by controlling for sources of imprecision, false positives, and false negatives; and,
identifying the subject is at risk of developing lung cancer if the physical data indicate that the levels in the biological sample are significantly different from the levels in the control.

In another aspect, there is described herein are methods to determine an actionable treatment recommendation for a subject diagnosed with lung cancer, comprising:
obtaining a biological sample from the subject detecting at least one feature that meets the threshold criteria for a positive value using a set of probes that hybridize to and amplify EGFR, ALK, ROS1, KRAS, BRAF, ERBB2, ERRBB4, MET, RET, FGFR1, FGFR2, FGFR3, DDR2, NRAS, PTEN, MAP2K1, TP53, STK1, CTNNB1, SMAD4, FBXW7, NOTCH 1, KIT/PGDFRA, PIK3CA, AKT1, and HRAS genes to detect the at least one feature that meets the threshold criteria for a positive value; and,
determining, based on the at least one feature with positive value detected, an actionable treatment recommendation for the subject.

In another aspect, there is described herein are methods of treatment for patients at risk of developing lung cancer wherein before medical management (e.g., screening for lung cancer and/or preventive treatment), risk of developing lung cancer is assessed by using any one of the kits as claimed herein; and,
the patients at low risk for developing lung cancer are subject to routine long term evaluation; and subsequently administering the medical treatment; and,
the patients at high risk of developing lung cancer or affected by lung cancer are subjected to preventive medical management or surgery for removing the lesions; and, subsequently administering the medical treatment.

In certain embodiments, measurement of low VAF mutants, comprises:
calculation of limit of detection/limit of quantification for measurement of each analyte in each specimen, based on measurement of specimen analyte relative to a known number of synthetic internal standard molecules.

In certain embodiments, the method comprises conducting the following steps:
step 1) multiplex gradient PCR to enable primers with varying melting temperatures to anneal to specific target;
step 2) single-plex PCR followed by quantification and equimolar mixing enables equal loading onto sequencer; and,
step 3) PCR targets chosen based on high occurrence in lung cancer and lung premalignant lesions.

In certain embodiments, the diagnosis or evaluation comprises one or more of a diagnosis of a lung cancer, a diagnosis of a stage of lung cancer, a diagnosis of a type or classification of a lung cancer, a diagnosis or detection of a recurrence of a lung cancer, a diagnosis or detection of a regression of a lung cancer, a prognosis of a lung cancer, or an evaluation of the response of a lung cancer to a surgical or non-surgical therapy.

In certain embodiments, the lung cancer is a non-small cell lung cancer.

In certain embodiments, the test subject has undergone surgery for solid tumor resection and/or chemotherapy, and/or radiation treatment.

In certain embodiments, the method further comprises a step where the patients are subjected to ongoing short-term evaluation.

In certain embodiments, the method further comprises a step where the patients are subjected to therapy with anti-cancer drugs.

In another aspect, there is described herein are uses of the kits and methods to facilitate approval by FDA and other regulatory agencies of lung cancer risk testing in kit or method form in regional laboratories.

In another aspect, there is described herein are uses of the kits and methods to facilitate approval by FDA and other regulatory agencies of testing for measurement of mutations in cancer cells that will then guide targeted therapy of the cancer in kit or method form in regional laboratories.

In another aspect, there is described herein are uses of the kits and methods to facilitate approval by FDA and other regulatory agencies of testing for measurement without unique molecular indices (UMI) of very low VAF (as low as 0.01%) mutations in cancer cells that will then guide targeted therapy of the cancer in kit or method form in regional laboratories.

In another aspect, there is described herein are uses of the kits and methods to enable measurement of lung cancer risk in non-invasively obtained specimens, such as exhaled breath condensate, bronchial brush and/or nasal brush specimens.

In another aspect, there is described herein are uses of the kits and methods to enable measurement of very low VAF mutations in airway epithelial cells.

In another aspect, there is described herein are uses of the kits and methods to measure mutations in cancer cells that will then guide targeted therapy of the cancer.

In another aspect, there is described herein are uses of the kits and methods to measure mutations in these genes in normal airway cells to determine risk for cancer.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A. Mutations identified in patient specimens. Sample mutation signal versus IS sequencing error. Variant allele frequency (VAF) of sample mutations (red triangle) relative to VAF of corresponding nucleotide-specific error variants in 19 IS replicates (black circle). VAF=site specific variant allele reads/total allele reads.

FIG. 1B. Showing how as the VAF % rises, there is a diminished difference between CA and NC subject, highlighting the importance of detecting variants with ultra-low VAF. It is likely that once a clone increases it's VAF to a significant size, the immune system takes it out. Thus, being able to identify low VAF clones allows for distinction between those at high risk for lung cancer and those at lower risk.

FIGS. 2A-2C. Inter-cohort comparison of TP53 mutation mean prevalence. FIG. 2A—Mean mutation prevalence among subjects within each cohort in each separate TP53 exon 5, 6, or 7 (mutations/target base/subject). FIG. 2B—Cohort- and substitution-specific mean mutation prevalence for the combined three TP53 exon targets. FIG. 2C—Number of mutations at TP53 hotspot sites. Inset: number of mutations according to mutation type. Mutations were defined as those with VAF (variant allele reads/total allele reads)>0.05% and significantly above IS background VAF based on contingency table analysis. TP53 mutations in CA-SMK subjects were enriched significantly at “hotspot” lung cancer driver mutation sites. (p=0.002).

FIGS. 3A-3B. Inter-cohort comparison of subject-specific mutation prevalence. Inter-cohort comparison of subject-specific mutation prevalence in (FIG. 3A) TP53 exons only or (FIG. 3B) TP53 exons, PIK3CA, and BRAF.

FIGS. 4A-4C. Inter-cohort comparison of EGFR mutation mean prevalence. FIG. 4A—Mean mutation prevalence among subjects within each cohort in each EGFR exon (18, 19, 20, or 21) (mutations/target base/subject). FIG. 4B—Cohort- and substitution-specific mean mutation prevalence for the combined four EGFR exon targets. FIG. 4C—Number of mutations at EGFR hotspot sites. Inset: number of mutations according to mutation type. Mutations were defined as those with VAF (variant allele reads/total allele reads) >5×1⁻⁴ (0.05%) and significantly above IS background VAF based on contingency table analysis.

FIG. 5. Qiagen CLC Genomics Workbench Settings.

FIG. 6. Schematic illustration of how to design internal standard (IS) spike-in molecules for NGS.

FIG. 7. Frequency of observed sequence variations for native template group and internal standards group for different types of sequence variations.

FIG. 8. Internal standard error for four replicates, showing the individual replicate error and mean error.

FIG. 9A. Hybrid capture panel for exons EGFR_18 (red), EGFR_20 (blue) and EGFR_21 (green), showing IS frequency (%).

FIG. 9B. NT frequency (%) showing replicate measurement, limit of blank (LOB), and variant allele frequency for exons EGFR_18 (red), EGFR_20 (blue) and EGFR_21 (green). Without internal standards, Limit of Blank (LOB) calculations are based on average error frequency across all variant types at all nucleotide positions. This effectively raises the Limit of Detection (LOD) and prevents statistical determination of variants with VAF<5%.

FIG. 9C Internal standards enable calculation of Limit of Blank (LOB) for each variant type at each nucleotide position providing site-specific determination of the Limit of Detection (LOD). This allows for identification of variants with VAF<1% at locations where the LOB is sufficient low.

FIG. 9D. Comparison of expected, NT, reported NT and reported IS for exons EGFR_18 (red), EGFR_20 (blue) and EGFR_21 (green).

FIG. 10. Applying Internal Standards to fragmented FDA Samples.

FIG. 11. Transition Sequencing Error at TP53 (exon 6) Across 19 Internal Standard Replicates, showing the Variant Allele Frequency for TP53 transactivation domain, TP53 DNA binding domain, and TP53 tetramerization domain.

FIG. 12. TP53 (exon 6) Transition Variants in Sample 7.

FIG. 13. Mutations in 19 Patient Specimens Relative to IS.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### Definitions and Abbreviations

AEC—Airway Epithelial Cells
CA-SMK—Cancer subjects, smokers
COSMIC—Catalog of Somatic Mutations in Cancer
FASMIC—Functional Annotation of Somatic Mutations in Cancer
FDA—Food and Drug Administration
HUGO—Human Genome Organization
IS—Internal Standard, synthetic DNA
ISM—Internal Standard Mixture
LCRT—Lung Cancer Risk Test
LDCT—Low Dose Computed Tomography
NC-NON—Non-cancer subjects, non-smokers
NC-SMK—Non-cancer subjects, smokers
NC-TOT—Non-cancer subjects, non-smokers+smokers (all non-cancer subjects)
NGS—Next Generation Sequencing
NT—Native Template, from targeted region of specimen DNA
PCR—Polymerase Chain Reaction
SNP—Single Nucleotide Polymorphism
VAF—Variant Allele Frequency
TCGA—The Cancer Genome Atlas

A “gene” is one or more sequence(s) of nucleotides in a genome that together encode one or more expressed molecules, e.g., an RNA, or polypeptide. The gene can include coding sequences that are transcribed into RNA which may then be translated into a polypeptide sequence, and can include associated structural or regulatory sequences that aid in replication or expression of the gene.

A “set” of markers, probes or primers refers to a collection or group of markers probes, primers, or the data derived therefrom, used for a common purpose (e.g., assessing an individual's risk of developing cancer). Frequently, data corresponding to the markers, probes or primers, or derived from their use, is stored in an electronic medium. While each of the members of a set possess utility with respect to the specified purpose, individual markers selected from the set as well as subsets including some, but not all of the markers, are also effective in achieving the specified purpose.

“Specimen” as used herein can refer to material collected for analysis, e.g., a swab of culture, a pinch of tissue, a biopsy extraction, a vial of a bodily fluid e.g., saliva, blood and/or urine, etc. that is taken for research, diagnostic or other purposes from any biological entity.

Specimen can also refer to amounts typically collected in biopsies, e.g., endoscopic biopsies (using brush and/or forceps), needle aspirate biopsies (including fine needle aspirate biopsies), as well as amounts provided in sorted cell populations (e.g., flow-sorted cell populations) and/or micro-dissected materials (e.g., laser captured micro-dissected tissues). For example, biopsies of suspected cancerous lesions, commonly are done by fine needle aspirate (FNA) biopsy, bone marrow is also obtained by biopsy, and tissues of the brain, developing embryo, and animal models may be obtained by laser captured micro-dissected samples.

“Biological entity” as used herein can refer to any entity capable of harboring a nucleic acid, including any species, e.g., a virus, a cell, a tissue, an in vitro culture, a plant, an animal, a subject participating in a clinical trial, and/or a subject being diagnosed or treated for a disease or condition.

“Sample” as used herein can refer to specimen material used for a given assay, reaction, run, trial and/or experiment. For example, a sample may comprise an aliquot of the specimen material collected, up to and including all of the specimen. As used herein the terms assay, reaction, run, trial and/or experiment can be used interchangeably

In some embodiments, the specimen collected may comprise less than about 100,000 cells, less than about 10,000 cells, less than about 5,000 cells, less than about 1,000 cells, less than about 500 cells, less than about 100 cells, less than about 50 cells, or less than about 10 cells.

In some embodiments, assessing, evaluating and/or measuring a nucleic acid can refer to providing a measure of the amount of a nucleic acid in a specimen and/or sample, e.g., to determine the level of expression of a gene. In some embodiments, providing a measure of an amount refers to detecting a presence or absence of the nucleic acid of interest. In some embodiments, providing a measure of an amount can refer to quantifying an amount of a nucleic acid can, e.g., providing a measure of concentration or degree of the amount of the nucleic acid present. In some embodiments, providing a measure of the amount of nucleic acid refer to enumerating the amount of the nucleic acid, e.g., indicating a number of molecules of the nucleic acid present in a sample. The “nucleic acid of interest” may be referred to as a “target” nucleic acid, and/or a “gene of interest,” e.g., a gene being evaluated, may be referred to as a target gene. The number of molecules of a nucleic acid can also be referred to as the number of copies of the nucleic acid found in a sample and/or specimen.

As used herein, “nucleic acid” can refer to a polymeric form of nucleotides and/or nucleotide-like molecules of any length. In certain embodiments, the nucleic acid can serve as a template for synthesis of a complementary nucleic acid, e.g., by base-complementary incorporation of nucleotide units. For example, a nucleic acid can comprise naturally occurring DNA, e.g., genomic DNA; RNA, e.g., mRNA, and/or can comprise a synthetic molecule, including but not limited to cDNA and recombinant molecules generated in any manner. For example the nucleic acid can be generated from chemical synthesis, reverse transcription, DNA replication or a combination of these generating methods. The linkage between the subunits can be provided by phosphates, phosphonates, phosphoramidates, phosphorothioates, or the like, or by nonphosphate groups, such as, but not limited to peptide-type linkages utilized in peptide nucleic acids (PNAs). The linking groups can be chiral or achiral. The polynucleotides can have any three-dimensional structure, encompassing single-stranded, double-stranded, and triple helical molecules that can be, e.g., DNA, RNA, or hybrid DNA/RNA molecules.

A nucleotide-like molecule can refer to a structural moiety that can act substantially like a nucleotide, for example exhibiting base complementarity with one or more of the bases that occur in DNA or RNA and/or being capable of base-complementary incorporation. The terms “polynucleotide,” “polynucleotide molecule,” “nucleic acid molecule,” “polynucleotide sequence” and “nucleic acid sequence,” can be used interchangeably with “nucleic acid” herein. In some specific embodiments, the nucleic acid to be measured may comprise a sequence corresponding to a specific gene.

In some embodiments the specimen collected comprises RNA to be measured, e.g., mRNA expressed in a tissue culture. In some embodiments the specimen collected comprises DNA to be measured, e.g., cDNA reverse transcribed from transcripts. In some embodiments, the nucleic acid to be measured is provided in a heterogeneous mixture of other nucleic acid molecules.

The term “native template” as used herein can refer to nucleic acid obtained directly or indirectly from a specimen that can serve as a template for amplification. For example, it may refer to cDNA molecules, corresponding to a gene whose expression is to be measured, where the cDNA is amplified and quantified.

The term “primer” generally refers to a nucleic acid capable of acting as a point of initiation of synthesis along a complementary strand when conditions are suitable for synthesis of a primer extension product.

### General Description

Described herein are kits and methods for assessing amounts of a nucleic acid in a sample. In some embodiments, the method allows measurement of small amounts of a nucleic acid, for example, where the nucleic acid is expressed in low amounts in a specimen, where small amounts of the nucleic acid remain intact and/or where small amounts of a specimen are provided.

### Design of Internal Standard (IS) Spike-In Molecules for NGS

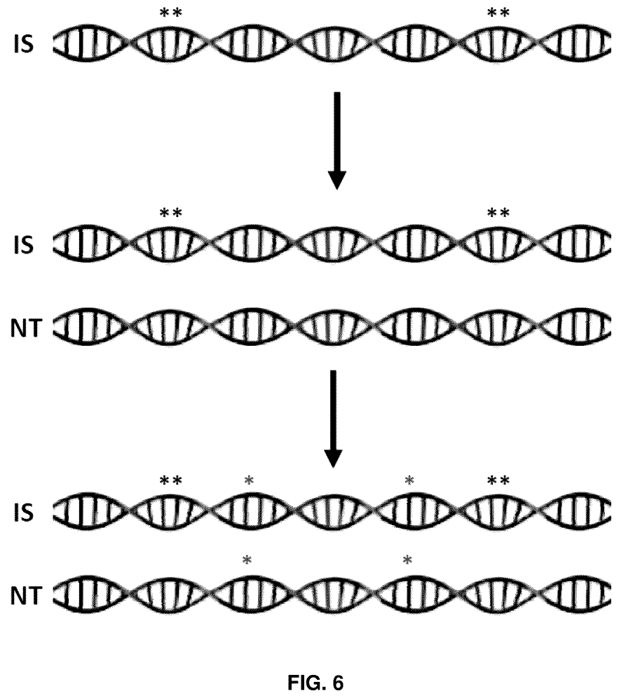

Referring first to FIG. 6, a schematic illustration of how to design internal standard (IS) spike-in molecules for NGS is shown.

IS are synthetic DNA molecule homologous with target analyte except for known one or more nucleotide changes.

IS Design goal: To behave the same as, but be distinguishable from target analyte DNA native template (NT)

IS Uses: 1) quantify measurable genome copies of each target analyte NT in library prep, and 2 quantify and characterize nucleotide site-specific technical error IS Implementation: 1) mix sample DNA with known number of IS molecules at 1:1 genome copy ratio prior to NGS library preparation; 2) co-amplify IS+NT mixture; 3) prepare sequencing library; and, 4) sequence sample.

Internal Standard “Spike-In Molecules” are custom perl script which separates IS reads from sample reads using one or more nucleotide changes. The error profile in native template (NT) nearly identical in internal standard (IS).

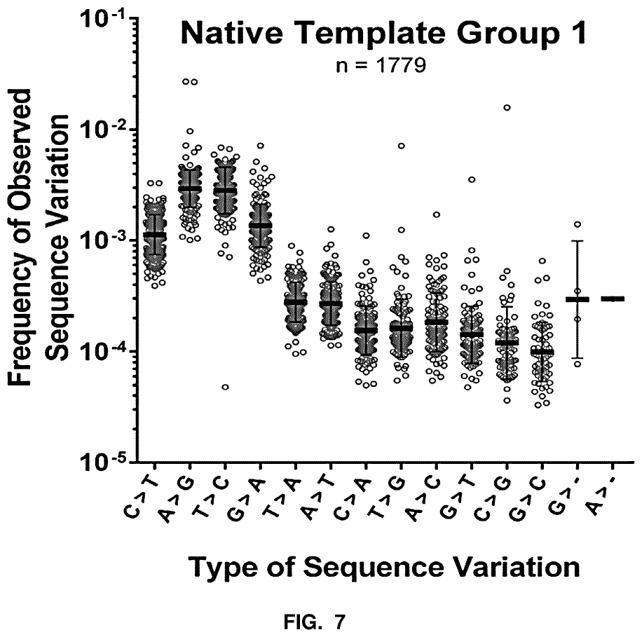

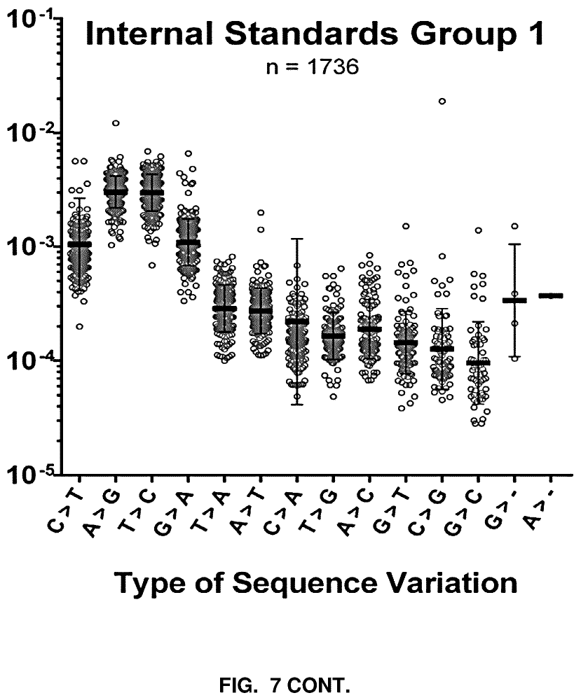

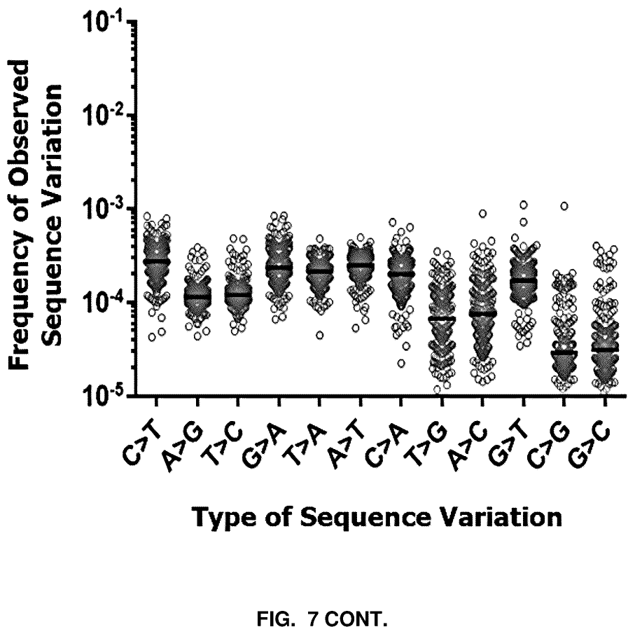

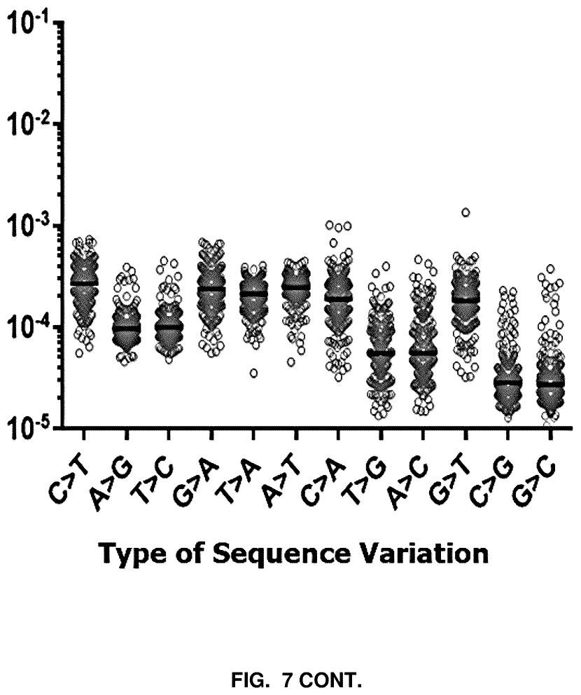

Thus, IS controls for library-specific error profiles, as shown in FIG. 7, which shows the frequency of observed sequence variations for native template group and internal standards group for different types of sequence variations.

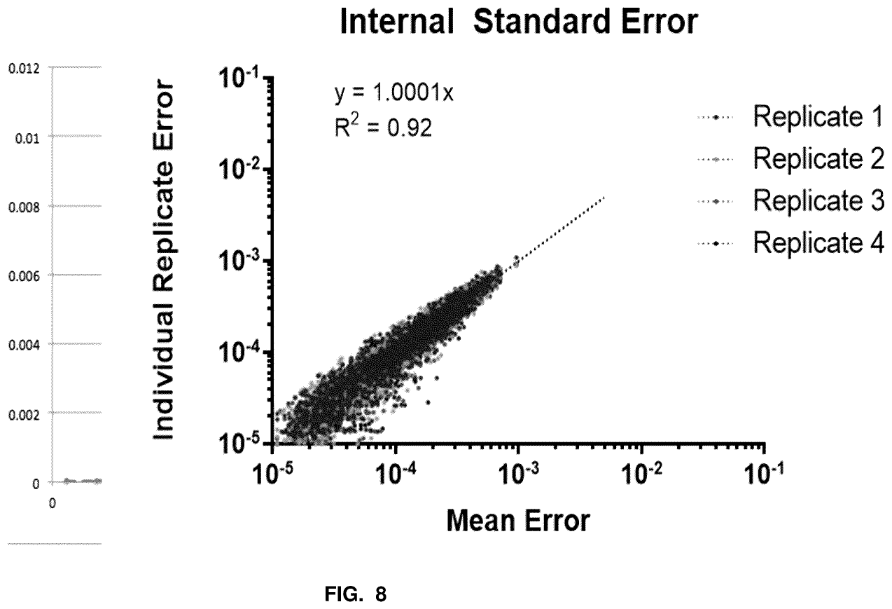

Additionally, as shown in FIG. 8, the nucleotide-specific technical error is reproducible. FIG. 8 shows the internal standard error for four replicates, showing the individual replicate error and mean error. The nucleotide-specific technical error at each NT base position matches corresponding IS position. Also, DNA landscape affects sequencing error on a region-to-region and nucleotide-to-nucleotide basis→IS and NT behave the same way.

Spiking IS into each reaction thus controls for variation within library prep (e.g., interfering substances, intra-and inter-panel hybridization efficiency, ligation efficiency, amplification).

Internal standards also control for sources of imprecision enabling narrow confidence interval at each nucleotide: nucleotide-specific error frequency; platform-specific errors, and polymerase-specific errors.

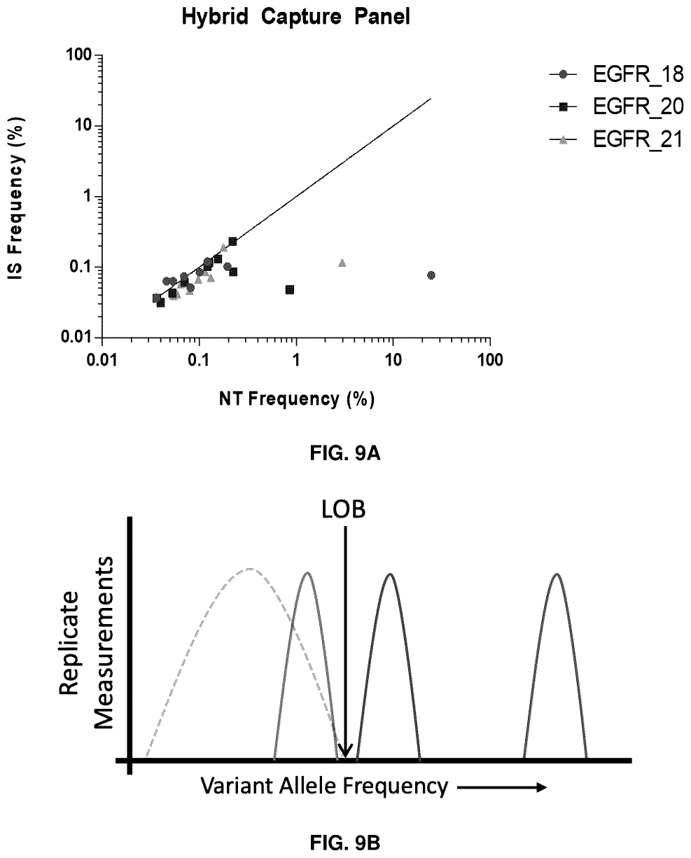

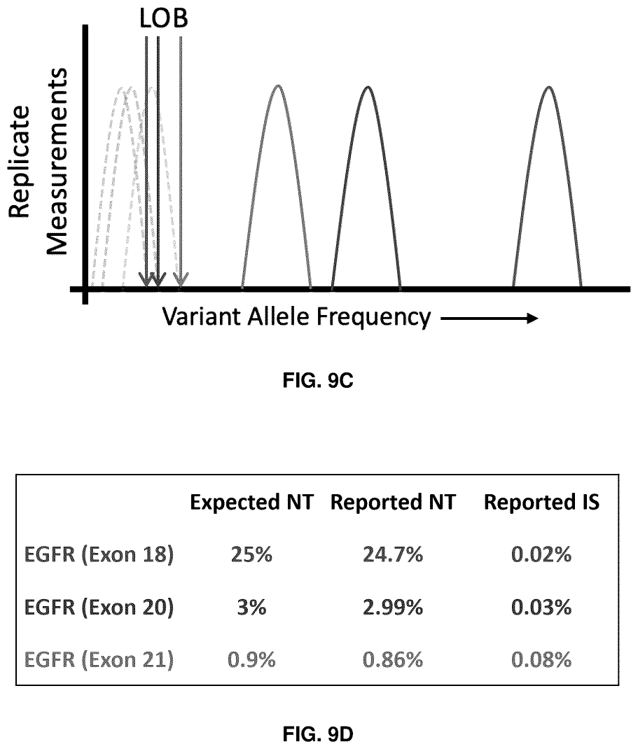

FIGS. 9A-9D show that internal standards enable site-specific LOD (logarithm of the odds). FIG. 9A shows a hybrid capture panel for exons EGFR_18 (red), EGFR_20 (blue) and EGFR_21 (green), showing IS frequency (%). FIGS. 9B-9C shows NT frequency (%), showing replicate measurement, LOB, and variant allele frequency for exons EGFR_18 (red), EGFR_20 (blue) and EGFR_21 (green). FIG. 9D shows a comparison of expected, NT, reported NT and reported IS for exons EGFR_18 (red), EGFR_20 (blue) and EGFR_21 (green). Thus, FIGS. 9A-9D show that traditional methods based on external process performance estimates do not support VAF measurements<5%. Also, alternative correction methods are complex and require 10- to 20 -fold more sequencing reads.

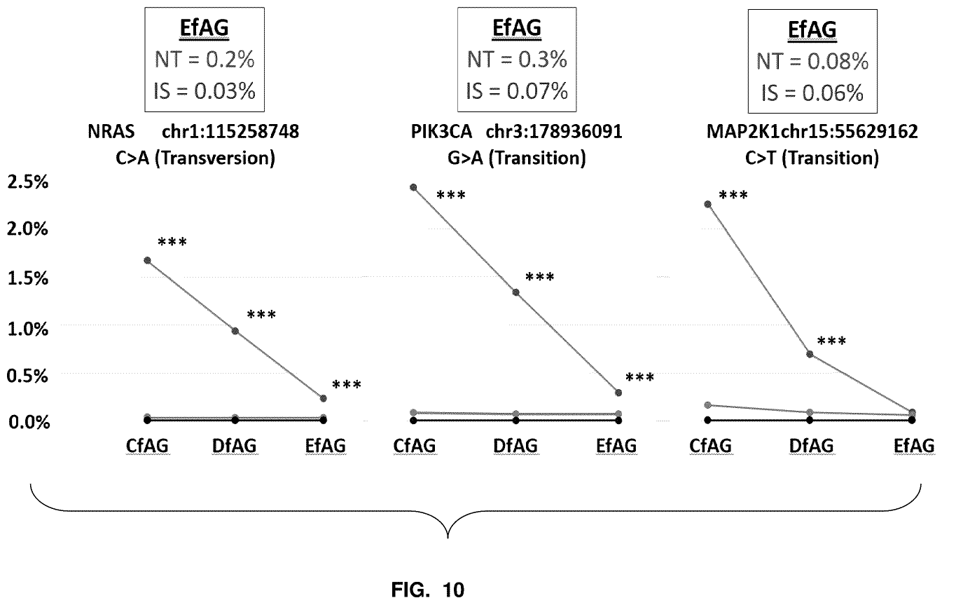

FIG. 10 shows applying Internal Standards (IS) to fragmented FDA samples. The known mutations identified with LOD based on site-specific LOB determined by internal standards (IS).

Multiplex gradient PCR enables primers with varying melting temperatures to anneal to specific target. Single-plex PCR followed by quantification and equimolar mixing enables equal loading onto sequencer. PCR targets chosen based on high occurrence in lung cancer and lung premalignant lesions.

Synthetic DNA internal standards (IS) were prepared for each of various lung cancer driver genes and mixed with each AEC genomic (gDNA) specimen prior to competitive multiplex PCR amplicon NGS library preparation. A custom Perl script was developed to separate IS reads and respective specimen gDNA reads from each target into separate files for parallel variant frequency analysis. This approach enabled reliable detection of mutations with VAF as low as 5×10⁻⁴ (0.05%). This method was then applied in a retrospective case-control study. Specifically, AEC specimens were collected by bronchoscopic brush biopsy from the normal airways of 19 subjects, including eleven lung cancer cases and eight non-cancer controls, and the association of lung cancer risk with AEC driver gene mutations was tested.

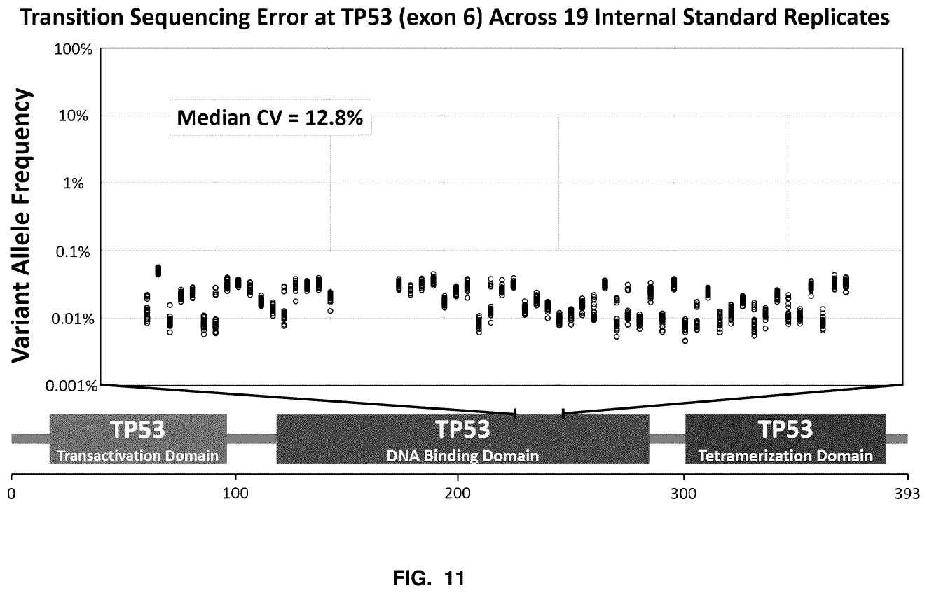

FIG. 11 is an example of transition sequencing error at TP53 (exon 6) across 19 Internal Standard(S) replicates, showing the variant allele frequency (VAF) for TP53 transactivation domain, TP53 DNA binding domain, and TP53 tetramerization domain.

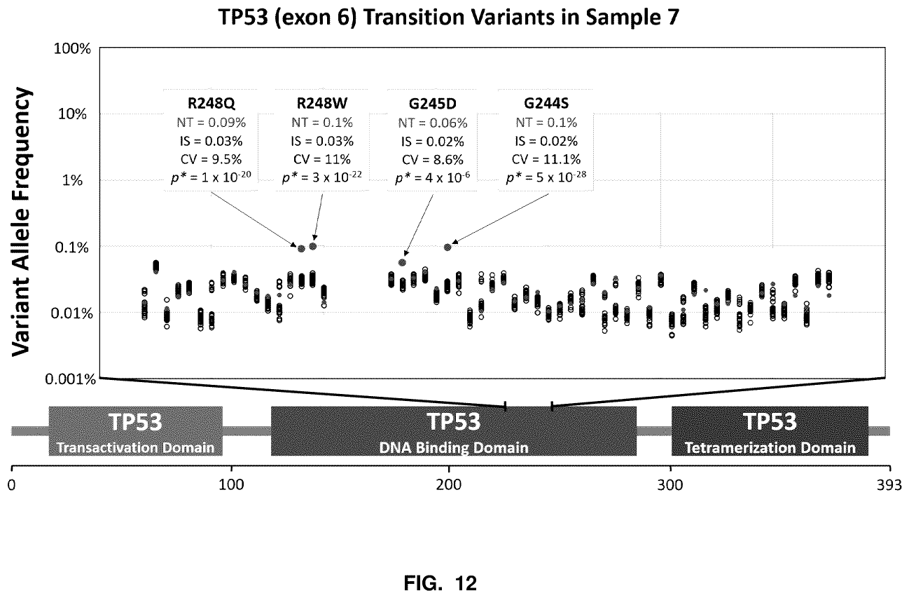

FIG. 12 is an example of transition variants in a sample at TP53 (exon 6), showing the variant allele frequency (VAF) for TP53 transactivation domain, TP53 DNA binding domain, and TP53 tetramerization domain.

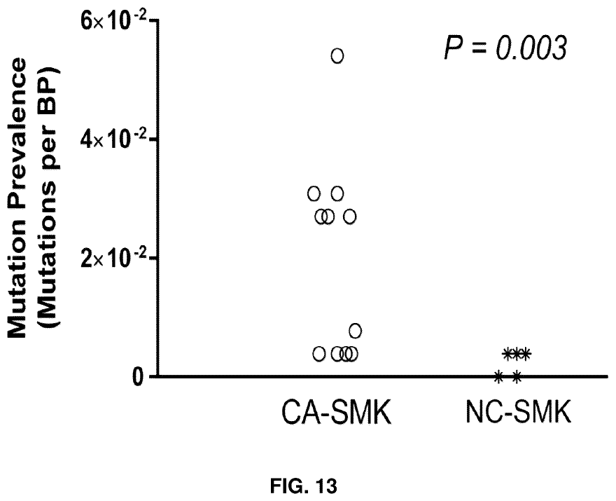

FIG. 13 shows mutations in 19 patient specimens relative to IS. 129 significant variants identified in 19 patient specimens. The VAF for these variants range from 0.05% to 0.46%. 99 variants found in 11 cancer specimens. 30 variants found in 8 non-cancer specimens. Also, there were significant increase in variants of smokers with cancer compared to smokers without cancer.

Described herein is a kit or method that includes reagents and instructions for measuring analytes in a lung cancer risk test.

This kit or method incorporates reagents for measurement of analytes that have not been previously described for inclusion in a test for lung cancer risk.

Specifically, the lung cancer risk test (LCRT) kit or method includes reagents for measurement of multiple low variant allele frequency (VAF) 55 i.e. VAF<0.01 II-0.0% l ) mutants in lung cancer driver genes, including TP53, PIK3CA, BRAF, KRAS, NRAS, NOTCHI, EGFR, and ERBB2.

Other reagents can be included for such genes as CDKN1A, E2F1, ERCC1, ERCC4, ERCC5, GPX1, GSTP1, KEAP1, RB1, TP53, TP63, and XRCC1.

These analytes may be measured in RNA or DNA from airway epithelial cells, and may be measured in non-invasively obtained specimens, including exhaled breath condensate and airway epithelial cells obtained by nasal brushings.

Also described herein are methods for measurement of low VAF mutants with calculation of limit of detection/limit of quantification for measurement of each analyte in each specimen, based on measurement of specimen analyte relative to a known number of synthetic internal standard molecules.

In certain embodiments, these kits and methods are useful to facilitate approval by FDA and other regulatory agencies of lung cancer risk testing in kit or method form in regional laboratories.

In certain embodiments, these kits and methods are useful to enable measurement of lung cancer risk in non-invasively obtained specimens, such as exhaled breath condensate, nasal brush specimens, sputum, oral epithelium, blood, and the like.

In certain embodiments, these kits and methods are useful to enable measurement of very low VAF mutations in airway epithelial cells.

### EXAMPLES

The methods and embodiments described herein are further defined in the following Examples, in which all parts and percentages are by weight and degrees are Celsius, unless otherwise stated. Certain embodiments of the present invention are defined in the Examples herein. It should be understood that these Examples, while indicating preferred embodiments of the invention, are given by way of illustration only. From the discussion herein and these Examples, one skilled in the art can ascertain the essential characteristics of this invention and without departing from the spirit and scope thereof, can make various changes and modifications of the invention to adapt it to various usages and conditions.

The measurement of mutations in the 0.05-1.0% VAF range enables more informative analysis of AEC somatic mutations associated with cancer risk. Among lung cancer subjects, TP53 mutations were more prevalent (p<0.05) and significantly more enriched for tobacco smoke and age signatures compared to non-cancer subjects matched for smoking and age.

### METHODS

### Study Cohort Enrollment and Characterization.

For this retrospective case-control study, AEC specimens collected from nineteen subjects were used, including eleven smokers with lung cancer (CA-SMK), five smokers without cancer (NC-SMK) matched for age and smoking history, and three non-smokers without cancer (NC-NON) (Table 1).

Subjects were enrolled into research trials at the University of Toledo Medical Center (UTMC) between 2000 and 2018. Each subject included in this research study provided written informed consent under protocols approved by the University of Toledo Institutional Review Board. Clinical characteristics, including lung cancer diagnosis, smoking history, and demographic information were obtained from the medical record. Lung cancer histology was reviewed and confirmed by an independent pathologist certified in anatomical and clinical pathology.

TABLE 1
Patient Demographics.

| Sample | Cancer | Pack | | | | Smoking | |
|---|---|---|---|---|---|---|---|
| # | Status | Years | Sex | Age | Race | Status | Diagnosis |
| 946 | CA | 45 | F | 55 | Black | Former | NSCLC- |
| | | | | | | | SQ |
| 167 | CA | 50 | F | 60 | Un- | Unknown | NSCLC |
| | | | | | known | | |
| 947 | CA | 45 | M | 61 | White | Former | SCLC |
| 146 | CA | 46.5 | F | 64 | White | Former | NSCLC |
| 887 | CA | 28 | F | 70 | White | Current | NSCLC- |
| | | | | | | | AD |
| 885 | CA | 90 | M | 73 | White | Current | SCLC |
| 940 | CA | 60 | M | 74 | White | Former | NSCLC- |
| | | | | | | | AD |
| 191 | CA | NA* | M | 75 | White | Current | NSCLC- |
| | | | | | | | SQ |
| 147 | CA | 75 | M | 76 | White | Former | SCLC |
| 128 | CA | 40 | F | 50 | Black | Current | NSCLC |
| 923 | CA | 15 | M | 79 | White | Former | NSCLC |
| 210 | NC | 34 | M | 40 | White | Current | Noncancer |
| 886 | NC | 0 | F | 46 | White | Never | Noncancer |
| 952 | NC | 30 | M | 52 | White | Former | Noncancer |
| 157 | NC | 100 | M | 60 | White | Unknown | Noncancer |
| 943 | NC | 0 | F | 65 | Black | Never | Noncancer |
| 956 | NC | 20 | M | 69 | Black | Current | Noncancer |
| 884 | NC | 54 | M | 77 | White | Former | Noncancer |
| 883 | NC | 0 | M | 81 | White | Never | Noncancer |

*Not available: The exact pack year smoking history for this patient was not recorded. However, it was recorded that the patient was an active 2 PPD smoker at time of lung cancer diagnosis at age 75 and had advanced stage COPD, thus there is compelling circumstantial evidence for large smoking history.

### Specimen Acquisition

AEC were obtained via bronchoscopic brush biopsy of normal appearing airway epithelium at the time of a diagnostic procedure done according to standard of care indication. For patients with a lung cancer diagnosis, sampling of AEC was from the main bronchus of the lung not involved with cancer. Specimens were immediately placed in cold saline and processed within one hour of collection.

### DNA Extraction and Quantification

Genomic DNA (gDNA) was extracted from approximately 500,000 AEC per subject using a FlexiGene DNA kit (Qiagen, Hilden, Germany) according to manufacturer protocol and quantified using competitive polymerase chain reaction (PCR) amplification of a well-characterized genomic locus in the Secretoglobin, family 1A, member 1 gene.

### Target Selection

Twelve loci in seven gene regions recently reported by The Cancer Genome Atlas (TCGA) project to be the most commonly mutated in non-small cell lung cancer were selected as targets. The targeted regions, specified according to Human Genome Organization (HUGO) names with exon numbers and abbreviations provided in parentheses, included B-Raf proto-oncogene exon 15 (BRAF_15), epidermal growth factor receptor exons 18-21 (EGFR_18, EGFR_19, EGFR_20, EGFR_21), erb-b2 receptor tyrosine kinase 2 (ERBB2), KRAS proto-oncogene exon 2 (KRAS_2), notch receptor 1 exon 26 (NOTCH1_26), phosphatidylinositol-4,5-bisphosphate 3-kinase catalytic subunit alpha exon 10 (PIK3CA_10), and tumor protein p53 exons 5-7 (TP53_5, TP53_6, TP53_7). Primers were developed for each of these targets.

Primers for all targets except for NOTCH1_26 performed efficiently in multiplex and downstream library preparation. As such, data are reported for the remaining 11 targets.

### Synthetic Internal Standard Mixture Preparation

Competitive synthetic DNA internal standard (IS) molecules for TCGA targets described above were designed with known dinucleotide substitution mutations relative to target analyte native template (NT) every 50 bases. This enabled separation of NT and IS reads during post-sequencing data processing of either PCR amplicon libraries used in this study, or of random fragment hybrid capture libraries in other ongoing studies not reported here. IS were cloned into plasmids and selected as pure clonal isolates using Sanger sequencing confirmation to verify the final sequence. This additional purification step was taken to select clones free of any potential errors introduced by synthesis. Due to the high fidelity of endogenous E. coli polymerase, the frequency of variants in the cloned IS can be expected to be between 10⁻⁷ to 10⁻⁸—well below the desired limit of detection for this study. Each cloned plasmid was linearized, quantified by digital droplet PCR, then combined in an equal genome copy balance. An internal standard mixture (ISM) containing equal concentrations (per genome copy) of each linearized target analyte IS molecule was prepared by Accugenomics, Inc. (Wilmington, NC).

Technically-derived base substitution errors occur at the same rate in synthetic IS as in the respective target sequence within gDNA test samples during the combined library preparation and sequencing steps. Therefore, each IS controls for target-specific site and regional differences in base substitution error rate.

### Multiplex Competitive PCR Amplicon Libraries

In order to amplify each target in a sample and maximize opportunity to detect low frequency variants, a multiplex competitive PCR amplicon library was prepared for each AEC DNA sample. Conditions were optimized to minimize technical error during PCR, including use of Q5 HotStart High Fidelity DNA Polymerase with a reported error frequency of 10⁻⁶ (New England Biolabs, Ipswich, MA) and minimization of PCR cycles in each round.

### Round 1: Competitive Multiplex PCR

Twelve target-specific primers with universal tails were synthesized by Life Technologies (Carlsbad, CA). Individual primer solutions for each target were created by adding TE buffer (10 mM Tris-Cl, pH 7.4, 0.1 mM EDTA) to the lyophilized primers to make a 100 μM stock. A 2.5 μM multiplex primer mixture was prepared by mixing 5 μL of each 100 μM forward and reverse primer stock solution and bringing the final volume to 200 μL with TE buffer.

For each subject, an aliquot of AEC DNA was combined with equal genome copies of ISM to control for nucleotide-specific substitution error occurring during library preparation and/or sequencing. Reactions containing at least 50,000 genome equivalents of both sample and IS in a mixture, 6 μL 5×Q5 Buffer (New England Biolabs, Ipswich, MA), 0.6 μL 10 mM dNTP (Promega, Madison, WI), 3 μL 2.5 μM multiplex primer mixture, 1.5 μL 2% w/v bovine serum albumin (New England Biolabs, Ipswich, MA), 0.3 μL Q5 HotStart High Fidelity DNA Polymerase (New England Biolabs, Ipswich, MA, Ipswich, MA), and molecular-grade water to a final reaction volume of 30 82 L were prepared.

Each competitive multiplex reaction mixture was amplified in a 7500 Fast Real-Time PCR System (Applied Biosystems, Foster City, CA) for a total of 20 cycles under modified gradient PCR conditions: 95° C./2 min (Q5 HotStart DNA Polymerase activation); 20 cycles of 94° C./10 sec (denaturation), 70° C./10 sec, 68° C./10 sec, 66° C./10 sec, 64° C./10 sec, 62° C./10 sec, (annealing), and 72° C./30 sec (extension); a final extension 72° C./2 min extension to ensure complete extension of all products. PCR products were column-purified using QIAquick PCR Purification Kit (Qiagen, Hilden, Germany) according to manufacturer protocol.

### Round 2: Singleplex PCR

Following multiplex amplification, a second round of 12 parallel singleplex PCR reactions using primers for each individual target at a final concentration of 500 nM were performed to ensure robust amplification of product for primers with lower efficiency in multiplex. High fidelity Q5 Hot Start Polymerase and other PCR reagents were used as described above.

Singleplex reactions were amplified in a 7500 Fast Real-Time PCR System (Applied Biosystems, Foster City, CA) for 15 cycles using the following conditions: 95° C./2 min (Q5polymerase activation); 15 cycles of 94° C./10 sec (denaturation), 65° C./20 sec, (annealing), and 72° C./30 sec (extension); a final extension 72° C./2 min extension was performed to ensure complete extension of all products. Each singleplex PCR product was checked for quality and quantity with an Agilent 2100 Bioanalyzer using DNA Chips with DNA 1000 Kit reagents according to manufacturer protocol (Agilent Technologies, Deutschland GmbH, Waldbronn, Germany). Sample-specific singleplex reactions then were (a) mixed in equimolar amounts to ensure an equal balance of target reads among sequencing read counts and (b) column-purified using QIAquick PCR Purification Kit (Qiagen, Hilden, Germany) according to manufacturer protocol.

### Round 3: Addition of Sample-Specific Barcodes

The column-purified mixture of singleplex reactions from each patient sample was labeled using a unique set of dual-indexed barcode primers to reduce likelihood of false-indexing/barcoding a sequencing read. A pair of fusion primers containing the barcode sequences and Illumina priming sites were designed with: 1) their 3′-end complementary to the universal sequence tails added during the initial multiplex and singleplex reactions, 2) 5′ to that a 10-nucleotide index/barcode sequence, and (3) 5′ to that, an Illumina Read 1 or Read 2 priming site.

The final concentration of the barcode primers in each reaction was 500 nM. PCR conditions were identical to those described for singleplex reactions except the cycle number was reduced to 10.

PCR products were checked for quality and quantity with an Agilent 2100 Bioanalyzer using DNA Chips with DNA 1000 Kit reagents according to manufacturer protocol and diluted 100-fold with molecular grade water for input into final sequencing adapter PCR.

### Round 4: Addition of Sequencing Adapters

Individual diluted barcoded samples were labeled with an Illumina platform-specific adapter using a second set of fusion primers designed with their 3′-end complimentary to the Illumina Read 1 or Read 2 priming sites and 5′ Illumina sequencing adapter using the same PCR conditions used in Round 3.

### Sample Pooling

Following Round 4, each uniquely barcoded sample was quantified on an Agilent 2100 Bioanalyzer as described above. The samples then were mixed in equimolar ratios to optimize the percentage of sequencing reads that each library would eventually receive; in most cases 1:1 was used.

### Product Purification and Sequencing

The combined sequencing library was purified using gel electrophoresis on a 2% w/v agarose gel. The resultant product band was then cut out, separating it from unwanted heterodimers, extracted using a QIAquick Gel Extraction Kit (Qiagen, Hilden, Germany), and eluted in 50 μL elution buffer. The purified sequencing library was sent to the University of Michigan Genomics core facility for Next Generation Sequencing on an Illumina NextSeq 550 sequencing

### Analysis of NGS Data

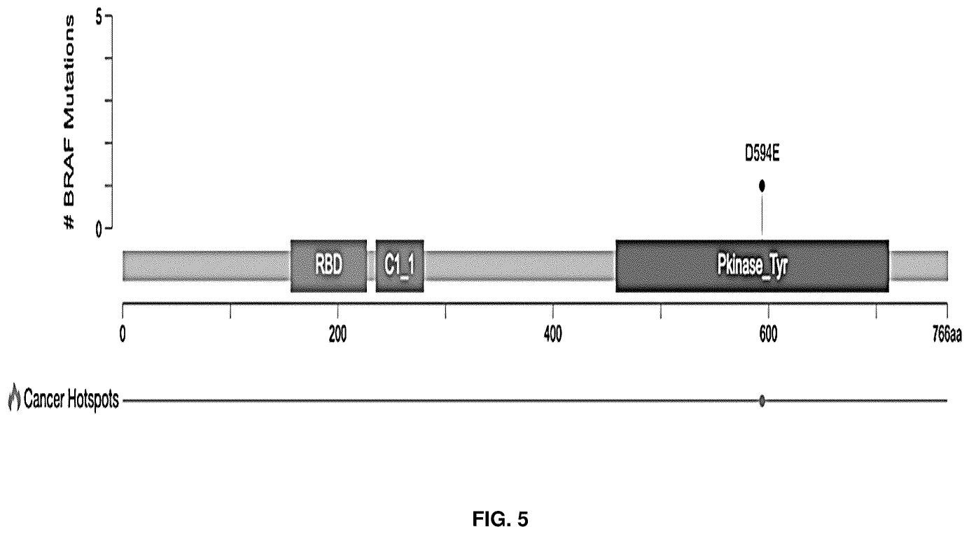

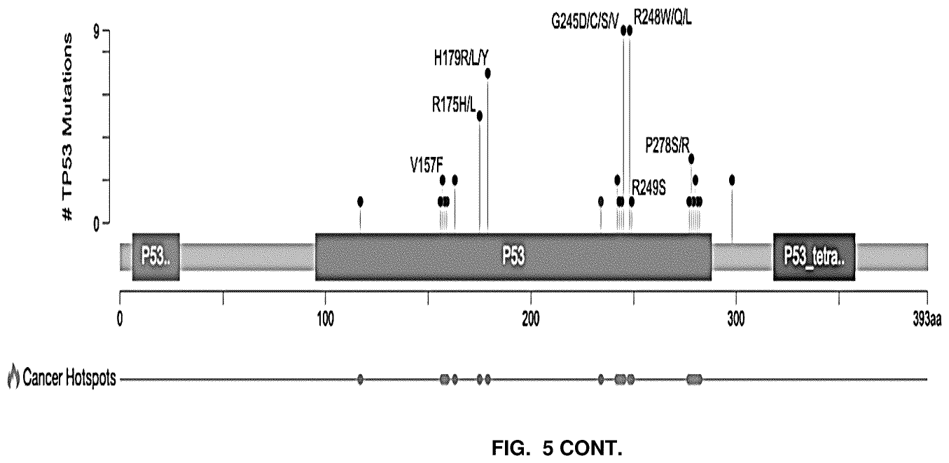

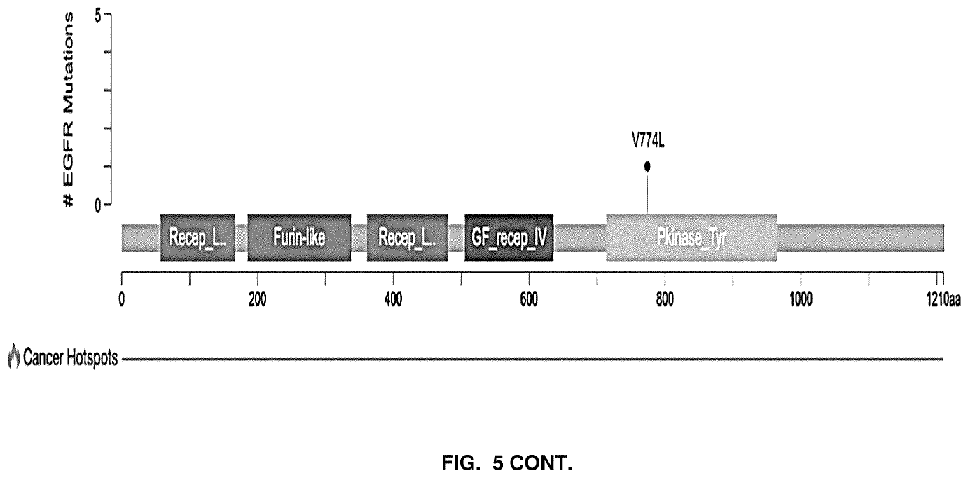

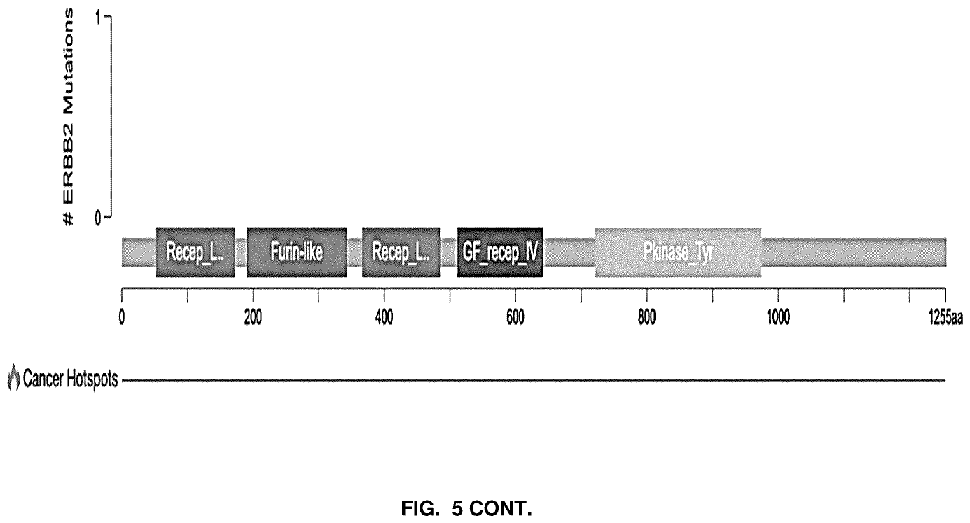

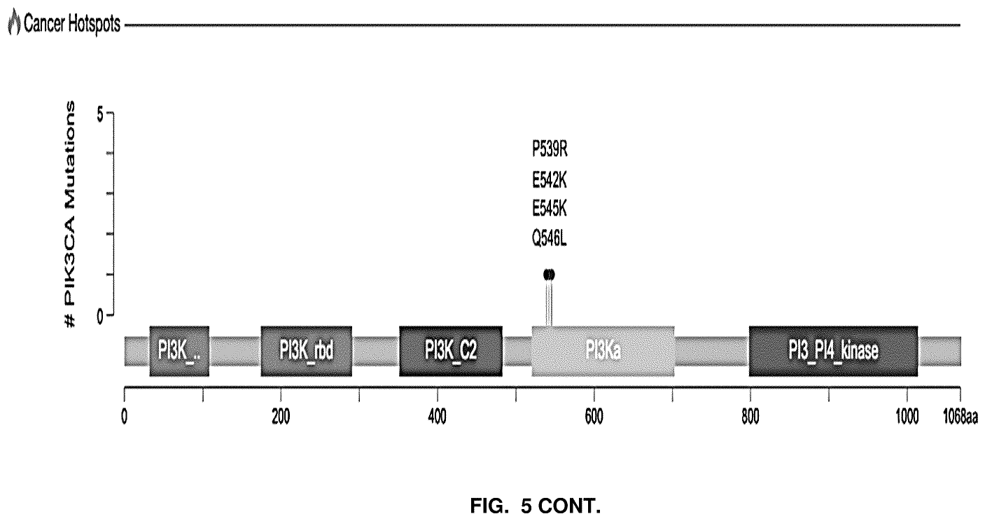

FASTQ data files generated by the University of Michigan Genomics core facility were processed using a custom Perl script to separate the internal standard (IS) and native template (NT) reads into separate NT and IS files, followed by parallel analysis using the Qiagen CLC Genomics Workbench 12 software suite for quality-trimming, alignment, and variant calling, as shown in FIG. 5.

Primer sequences, internal standard dinucleotide positions plus their 5′ and 3′ bases, and known single nucleotide polymorphism (SNP) positions were excluded from variant analysis.

### Variant Calling

Variants were called based on NT signal significantly above the background error measured in IS for the respective mutation type at each respective position. Significance was determined using contingency table chi-square analysis of each individual variant type at each nucleotide position, for identifying rare variants in pooled samples. To maximize stringency of test for signal above noise, a variant was called if the proportion of variant reads to wild-type reads in the specimen was significantly higher than the proportion of variant reads to wild-type reads at the same site in the IS mixed with the respective specimen, and also higher than the proportion observed in IS mixed with each of the other 18 specimens. Thus, each variant in a specimen was considered a true positive (p<0.05) only if the proportion of variant reads to wild-type reads was significantly higher in the specimen than each of the 19 IS replicates. A Bonferroni correction for false discovery was used based on the number of nucleotides assessed (760 bp) and the number of substitution mutations possible at each nucleotide position. Further, to avoid potential analytical variation from stochastic sampling, only mutations with significant signal above IS noise, and with VAF>0.05% were called.

### Variant Annotation and Hotspot Analysis

Called variants were characterized for pathogenicity using publicly-available databases including dbSNP, COSMIC, and FASMIC. Identification of known oncogenic hotspots and generation of corresponding figures were assessed using the cBioPortal for Cancer Genomics developed at Memorial Sloan Kettering (MSK) Cancer Center.

### Statistical Analysis

Calling of variants based on contingency table chi-square analysis of each individual variant type at each nucleotide position was performed using R: A Language and Environment for Statistical Computing (www.R-project.org/). Assessment of hotspot enrichment for called variants was performed using Kruskal-Wallis test using a chi-square distribution. Mutation prevalence based on type of mutation and target was assessed using Kruskal-Wallis test with Nemenyi test for multiple comparisons.

### RESULTS

### Measurement of Low Frequency Mutations in Non-Cancer Aairway Epithelium

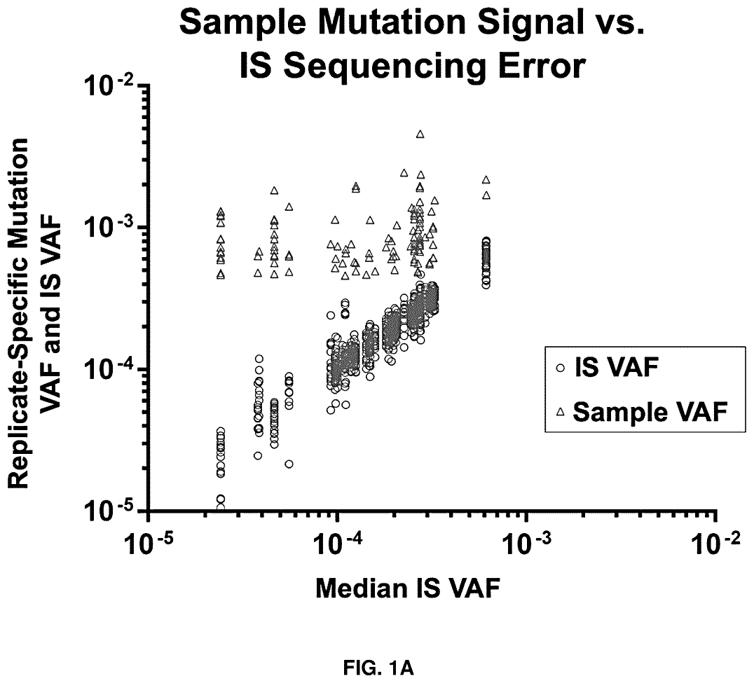

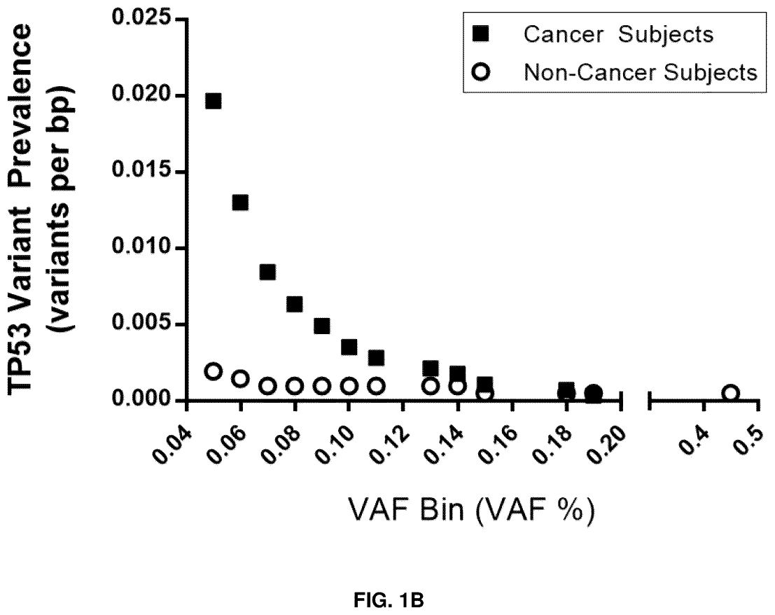

In this study of 11 driver gene target regions in AEC specimens from normal airways of 19 subjects, there were 129 called variants with VAF ranging from 5×10⁻⁴ (0.05%) to 4.6×10⁻³ (0.46%). As described in the Methods section, a VAF minimum threshold of 0.05% was used to minimize risk of false discovery due to stochastic sampling. Among the 129 called variants, the relationship between sample mutation signal (Mutation VAF) and background technical error (noise) (IS VAF) for the respective variant at the same site is presented in FIG. 1A.

For each sample mutation VAF, there is displayed the IS VAF for 19 IS. These represent the VAF for the IS mixed with the sample that contained the mutation as well as the VAF for each of the IS mixed with the other 18 samples. These 19 independent IS replicate values display the variation around the IS VAF (error) measurement within an experiment. The inter-replicate variation in IS VAF values increases with decreasing IS VAF, consistent with effects of the Poisson distribution on stochastic sampling.

Further, due to very low technical error at some of these sites there was no IS VAF value (FIG. 1A).

These effects of Poisson distribution present challenges for statistical analysis of significance for observed sample mutations. A simple Z-score analysis is appropriate if there are at least 10 sequencing reads for all four components: sample reference and variant allele, and IS reference and variant (error) allele. Using a minimum sample mutation VAF of 0.05% ensured at least 10 variant allele reads for each called sample mutation. However, when the corresponding IS error was very low, the IS variant allele read count was below 10, and sometimes zero.

If there were at least one variant allele read for each IS replicate, it would be appropriate to use Poisson exact test. In this study, because the IS error in the targeted hotspot regions was so low that for some measurements there were zero IS variant reads corresponding to observed sample variants, even with the deep sequencing employed it was advantageous to use the contingency table approach to determine significance of each sample mutation in this study.

FIG. 1B shows TP53 mutations detectable in AEC depends on lower limit of detection for VAF (%) detection.

A key reason that a TP53 mutation test for lung cancer risk measured in airway epithelial cells was not discovered previously, in spite of efforts to do so is that commonly used methods are not able to reliably measure mutations at VAF<1%.

### Characteristics of Sequencing Error in the Targeted Regions

As shown in FIG. 1A, the maximum sequencing error (Median IS VAF across replicates) at sites within the targeted regions for which a sample variant was called was 0.06%. This error rate is much lower than that observed for whole exome sequencing on Illumina platform. In addition, as reported by others, this is a key factor that enables meaningful calling of low frequency variants without need for methods that employ unique molecular indices (UMI) with attendant cost and computational requirements.

### Prevalence of Low Frequency Mutations in AEC

Mutation prevalence was calculated as called mutations per nucleotide positions assessed for each target. The number of nucleotides assessed for each target varied somewhat based on region spanned by primers and number of dinucleotide sites blocked from analysis due to modification in IS to enable separation of IS reads from NT reads. Among all 19 subjects, the average mutation prevalence, across the targeted DNA region (760 bp) in each subject (mutations/bp/subject) was 8.9×10⁻³. (Table 2).

TABLE 2
Target- and cohort-specific mutation prevalence.

| | | | | | Average |
|---|---|---|---|---|---|
| | | | | | (All |
| Target | CA-SMK | NC-SMK | NC-NON | NC-TOT | Subjects) |
| BRAF_15 | 6.7 × 10⁻³ | 0 | 0 | 0 | 3.9 × 10⁻³ |
| EGFR_18 | 0 | 0 | 0 | 0 | 0 |
| EGFR_19 | 0 | 0 | 0 | 0 | 0 |
| EGFR_20 | 3.9 × 10⁻² | 3.4 × 10⁻² | 4.5 × 10⁻² | 3.8 × 10⁻² | 3.8 × 10⁻² |
| EGFR_21 | 1.7 × 10⁻³ | 0 | 0 | 0 | 9.9 × 10⁻⁴ |
| ERBB2 | 1.1 × 10⁻² | 1.4 × 10⁻² | 1.4 × 10⁻² | 1.4 × 10⁻² | 1.2 × 10⁻² |
| KRAS_2 | 0 | 0 | 0 | 0 | 0 |
| PIK3CA_10 | 4.2 × 10⁻³ | 0 | 0 | 0 | 2.4 × 10⁻³ |
| TP53_5 | 2.2 × 10⁻² | 4.7 × 10⁻³ | 0 | 2.9 × 10⁻³ | 1.4 × 10⁻² |
| TP53_6 | 2.2 × 10⁻² | 0 | 3.1 × 10⁻³ | 1.2 × 10⁻³ | 1.3 × 10⁻² |
| TP53_7 | 1.3 × 10⁻² | 2.9 × 10⁻³ | 0 | 1.8 × 10⁻³ | 8.5 × 10⁻³ |
| Average | 1.2 × 10⁻² | 4.7 × 10⁻³ | 5.3 × 10⁻³ | 4.9 × 10⁻³ | 8.9 × 10⁻³ |
| (All Targets) | | | | | |

This AEC mutation prevalence value is much higher than reported for methods that only detect mutants with relatively high variant frequency (VAF>1%) (14), or that are more sensitive but non-targeted. However, it is consistent with other analysis of AEC using a highly sensitive PCR-based method.

Association of Low Frequency Substitution Mutations in TP53, PIK3CA, and BRAF with Lung Cancer

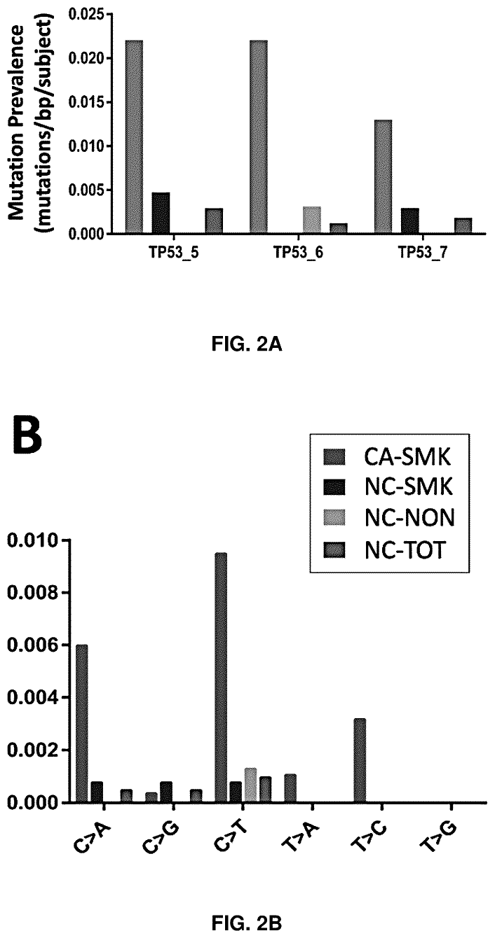

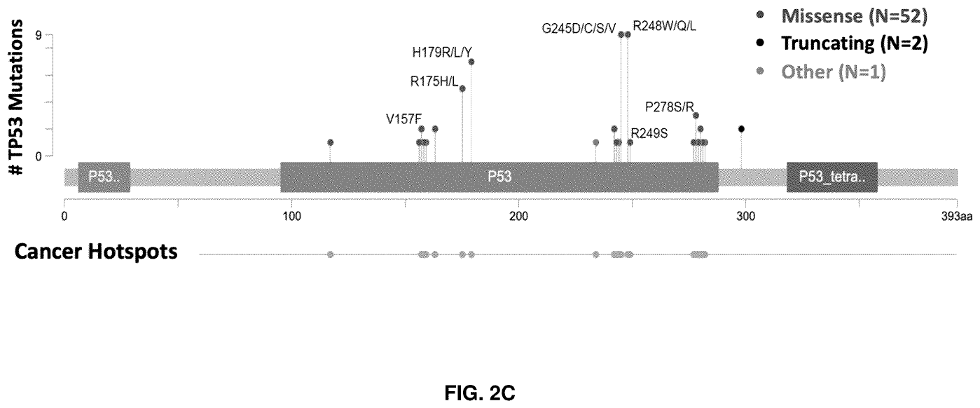

Among the three measured exons of TP53, the prevalence (mutations/bp/subject) of substitution mutations was 10.4-fold higher (p<0.05) in AEC from CA-SMK subjects relative to NC-SMK subjects matched for smoking and age (FIG. 2A, Table 3).

TABLE 3
Statistical analysis of target specific inter-
cohort differences in mutation prevalence.

| | CA-SMK vs. | CA-SMK vs. | CA-SMK vs. | NC-SMK vs. |
|---|---|---|---|---|
| Target | NC-TOT | NC-SMK | NC-NON | NC-NON |
| BRAF_15 | 0.12 | 0.4 | 0.54 | 1 |
| EGFR_18 | N/A | N/A | N/A | N/A |
| EGFR_19 | N/A | N/A | N/A | N/A |
| EGFR_20 | 0.72 | 0.78 | 0.96 | 0.74 |
| EGFR_21 | 0.39 | 0.76 | 0.83 | 1 |
| ERBB2 | 0.35 | 0.73 | 0.8 | 1 |
| KRAS_2 | N/A | N/A | N/A | N/A |
| PIK3CA_10 | 0.062 | 0.27 | 0.41 | 1 |
| TP53_5 | 0.022 | 0.27 | 0.1 | 0.77 |
| TP53_6 | 0.0083 | 0.037 | 0.333 | 0.849 |
| TP53_7 | 0.028 | 0.25 | 0.16 | 0.9 |
| TP53_Total | 0.0019 | 0.047 | 0.043 | 0.92 |

In addition, PIK3CA or BRAF mutations were observed in seven cancer subjects and no non-cancer subjects (Table 3).

Notably, the majority of mutations in TP53 (FIG. 2C), all of the mutations in PIK3CA, and one of three mutations in BRAF occurred in previously identified “hotspots” associated with biological changes that drive carcinogenesis.

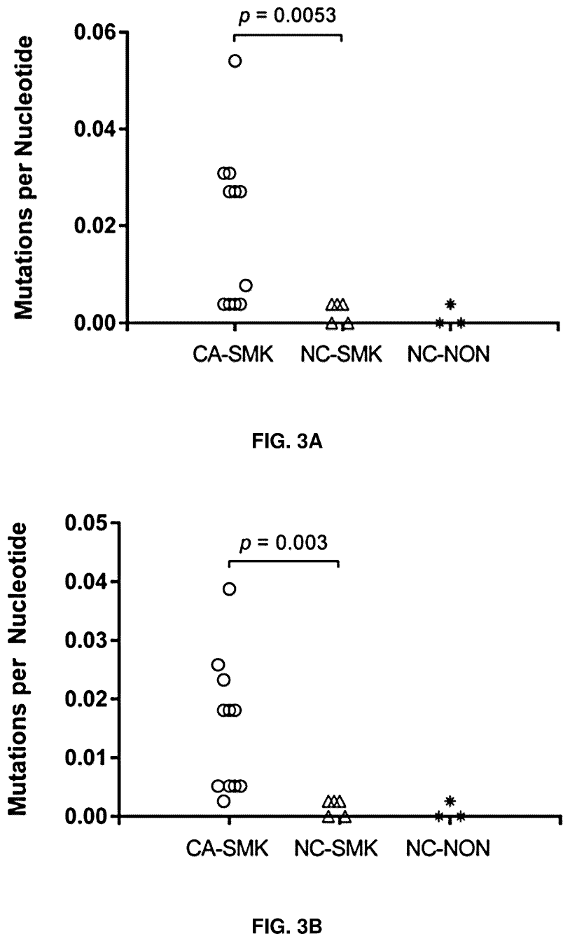

Toward the goal of developing a biomarker that might contribute to improved determination of lung cancer risk, we assessed subject-specific inter-cohort differences in prevalence of these low frequency mutations. Based on data obtained in this small retrospective case-control study, a TP53 exon mutation prevalence cut-off of 0.02 mutations/bp would have 100% specificity and 55% sensitivity (FIG. 3A). Similar discrimination was observed when TP53 exon mutations were combined with PIK3CA, and BRAF mutations (FIG. 3B).

Nearly all of the TP53 mutations in CA-SMK subjects were tobacco signature or age-related mutations (C>A, C>T, and T>C substitutions) (FIG. 2B, Table 4), closely approximating the spectrum of TP53 mutations reported for lung cancer tissues. The prevalence of each type of tobacco or age signature TP53 mutation was significantly higher in cancer subjects than in non-cancer subjects, including C>A (p=0.002), C>T (p=0.003), and T>C (p =0.001) (Table 4).

For example, while C to A mutations comprised 29.8% (17/57) of TP53 mutations observed in AEC from CA-SMK subjects, there was only one C to A TP53 mutation observed in all non-cancer subjects (NC-TOT) (Table 4). C>T transitions comprised 47% of TP53 mutations in lung cancer subjects in this study. Further, TP53 mutations in CA-SMK subjects were enriched significantly (p=0.002) at “hotspot” lung cancer driver mutation sites (FIG. 2C).

TABLE 4
Inter-cohort comparison of type-specific substitution
mutations across all TP53 exons.

| Mutation | CA-SMK¹ | NC-SMK² | NC-NON³ | NC-TOT⁴ |
|---|---|---|---|---|
| C > A | 17 (2.0 × 10⁻³)* | 1 (1.2 × 10⁻⁴) | 0 | 1 (1.2 × 10⁻⁴) |
| C > G | 1 (1.2 × 10⁻⁴) | 1 (1.2 × 10⁻⁴) | 0 | 1 (1.2 × 10⁻⁴) |
| C > T | 27 (3.2 × 10⁻³)*** | 1 (1.2 × 10⁻⁴) | 1 (1.2 × | 2 (2.4 × 10⁻⁴) |
| | | | 10⁻⁴) | |
| T > A | 3 (3.6 × 10⁻⁴) | 0 | 0 | 0 |
| T > C | 9 (1.1 × 10⁻⁴)* | 0 | 0 | 0 |
| T > G | 0 | 0 | 0 | 0 |

¹CA-SMK; Cancer subject, present or past smoker.
²NC-SMK; Non-Cancer subject, present or past smoker.
³NC-NON; Non-Cancer subject, never smoker.
⁴NC-TOT; All Non-Cancer subjects, smokers and non-smokers.
*p < 0.05;
**p < 0.01;
*** p < 0.005

Lack of Association of TP53 Mutations with Smoking History

Notably, among non-cancer subjects, smoking was not associated with higher TP53 mutation prevalence (Table 3). Specifically, only half of NC-SMK subjects had a TP53 mutation with VAF>0.05% and in each case, only one variant was observed. (Table 3). Due to the small number of PIK3CA and BRAF mutations it was not possible to address a smoking association. Characteristics of Low Frequency AEC Mutations Not Associated with Lung Cancer

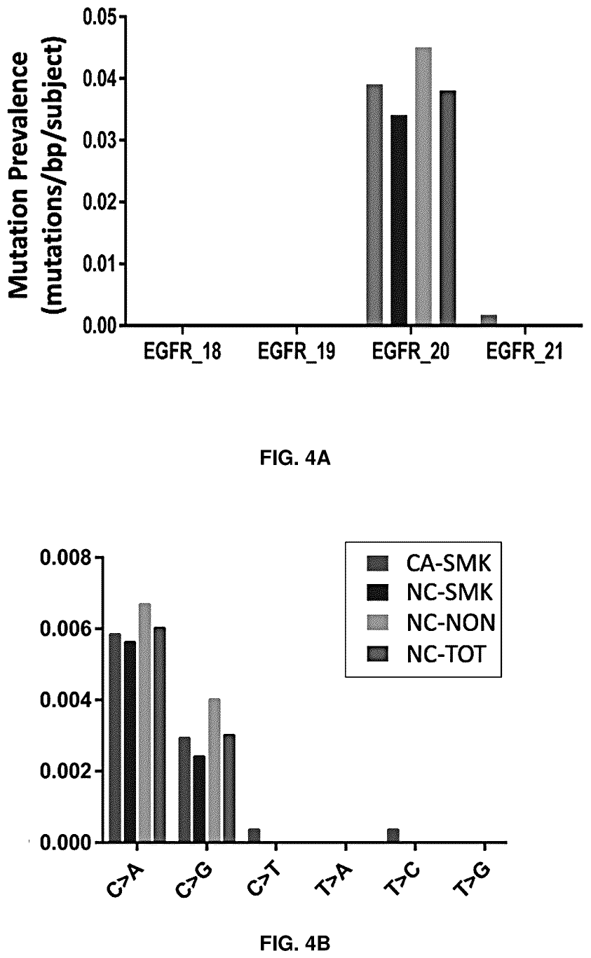

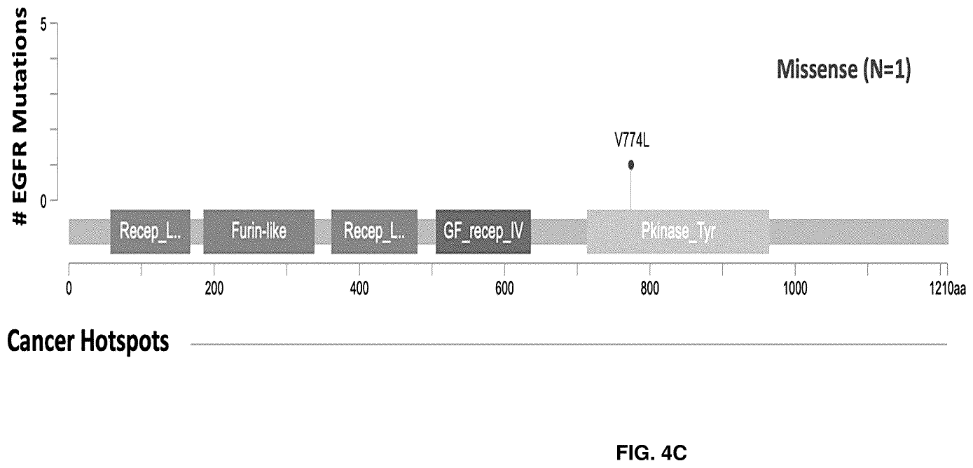

In contrast to TP53, at non-TP53 targets the mutation prevalence was not significantly different in cancer compared to non-cancer subjects (Table 3). Among the 11 targets measured, mutation count was highest in the EGFR_20 target region with a total of 43 mutations observed across all subjects (Table 3). There was no difference in EGFR_20 mutation prevalence between cancer and non-cancer (3.9×10^{-2 vs} 3.8×10⁻², respectively; p=0.72) (FIG. 4A, Table 3), and no association between smoking and non-smoking (3.4×¹⁰⁻² vs 4.5×10⁻² respectively; p=0.74). ERBB2 mutations (N=17) displayed a similar spectrum to that of EGFR_20 with no age or tobacco signature mutation pattern and no difference among the cohorts. Notably, in contrast to the high fraction of C>T transitions among TP53 (29/61; 48%), only 1/43 (2.3%) EGFR_20 mutations, and 1 ERBB2 mutation was C>T (FIG. 3B). Further, the majority of the EGFR_20 mutations were synonymous and not predicted to be pathogenic (FIG. 3C).

### DISCUSSION

### Measurement of Low Frequency Mutations in AEC

The ability to measure low frequency mutations in AEC in this study was due to a combination of low technical error in the regions targeted (FIG. 1), and the use of synthetic internal standards to control for technical error on a site-and variant-specific basis (FIG. 1). The range of prevalence for low frequency TP53 mutations in AEC among subjects in this study was similar to previously reported. The enrichment for TP53 mutations in driver mutation sites and for tobacco-smoke signatures provides another source of validation that the observed mutations are true positives.

Identification of a TP53 Mutation Field Effect Associated with Lung Cancer Risk

The higher prevalence of low frequency TP53 hot-spot pathogenic tobacco smoke and age signature mutations in AEC of CA subjects compared with NC subjects matched for smoking and age represents a field of injury strongly associated with lung cancer risk (FIG. 2A, FIG. 2B, FIG. 3A, Table 3, Table 4).

Thus, the results of low frequency (i.e., VAF<1%) show that TP53 hotspot mutations in AEC are a lung cancer risk biomarker. Moreover, inclusion of low frequency actionable mutations in BRAF and PIK3CA can further enhance accuracy of this biomarker (FIG. 3B).

Lung cancer predisposition is due, in part, to sub-optimal protection from DNA damage associated with cigarette smoking and age-related DNA replication errors. There is evidence for both hereditary and acquired causes of sub-optimal AEC protection from DNA damage. For example, there is a large inter-individual variation in regulation of key DNA repair, antioxidant, and cell-cycle control genes in AEC, and the lung cancer risk test (LCRT) based on this variation, has high accuracy to identify lung cancer subjects.

One of the variables in the LCRT biomarker is TP53 transcript abundance, and there is a 100-fold variation in TP53 expression in AEC. TP53 plays a key role in upregulating DNA repair genes in response to DNA damage, and the TP53 protein directly regulates the key nucleotide excision repair (NER) gene, ERCC5, in AEC.

The germ line allelic variation at rs2296147, a TP53 recognition site in the 5′-regulatory region of ERCC5, is associated with variation in allele-specific expression of ERCC5 in AEC. Hereditary inter-individual variation in ERCC5 transcription regulation by TP53 is significant because ERCC5 is the rate-limiting enzyme in transcription-coupled NER, and mutations associated with tobacco smoke result from inefficient NER of DNA adducts arising from the binding of cigarette smoke carcinogen metabolites to the exocyclic N2-positions of guanines on the transcribed strand.

Thus, sub-optimal ERCC5 regulation by TP53, determined by inherited germ line variants, is an important factor responsible for higher prevalence of tobacco smoke induced hotspot mutations in the transcribed strand of TP53 among cancer subjects.

### Interpretation of Non-Pathogenic EGFR Mutations

There was no difference in prevalence between cancer and non-cancer subjects or smokers and non-smokers for EGFR total mutations or cigarette-or age-signature mutations (FIG. 4A, FIG. 4B; Table 2, Table 3). The substitution pattern (evenly distributed between C>A and C>G) is most consistent with previously described Signature 3, associated with sub-optimal homologous-recombination DNA double-strand break repair. In addition, evidence presented here supports the conclusion that the observed EGFR exon 20 mutations do not confer growth advantage.

Specifically, in contrast to the observed non-synonymous pathogenic TP53 smoke- and age-related mutations, only 1/43 EGFR_20 mutations was synonymous and present at a known pathogenic hotspot (FIG. 4C).

It is now believed that clonal populations with this type of mutation likely occurred as stochastic DNA replication errors in stem cell proliferation to generate the airway epithelium during the fetal-juvenile period.

A highly sensitive mismatch PCR assay capable of detecting VAF as low as 5×10⁻⁵ (0.005%) was used to test for the effect of cigarette smoke on prevalence of low VAF somatic mutations in AEC of non-cancer patients, including mutations in TP53, KRAS, and HPRT1 genes. Surprisingly, among these non-cancer subjects, there was no effect of smoking on the prevalence of TP53 or KRAS mutations in AEC.

It is also now believed that in individuals without lung cancer, either smoker or non-smoker, most low frequency mutations in airway epithelium are the consequence of cell replication-related stochastic mutation events that occur during tissue development in the fetal/neonatal period.

### Biomarkers for Targeted Chemoprevention

Currently, there is no targeted therapy for lung cancer-associated TP53 mutations. However, mutations at lung cancer-associated PIK3CA or BRAF hotspots were detected in the AEC of six of the 11 lung cancer subjects and none of the non-cancer subjects (Table 3). For each subject in this study, DNA was extracted from approximately 500,000 AEC, and for each of the six subjects positive for PIK3CA or BRAF mutations, the average mutation VAF was about 10-3.Thus, if clones were evenly distributed at a similar prevalence, using a prior estimation of 5×10⁸ AEC throughout bronchial trees of both lungs, it would be expected that a total of 10⁵ mutations in 1,000 colonies per subject. Relatively non-toxic gene targeted therapies for PIK3CA and BRAF are FDA-approved or in advanced trials for some cancers. For example, alpelisib is currently in Phase III trials for treatment of PIK3CA driver mutations in cancers of the lung and other tissues, and a combination of dabrafenib and trametinib has clear efficacy in treatment of BRAF: V600E mutated non-small cell lung cancers.

Thus, the presently described test of PIK3CA/BRAF prevalence in AEC is useful where the AEC mutation spectrum is measured before and after treatment of lung cancer subjects bearing cancers. Thus, well-tolerated gene targeted therapy could reduce the burden of AEC field of injury mutations that contribute to development of lung cancer. Then, individuals with elevated PIK3CA/BRAF mutation prevalence in AEC could be considered for chemoprevention trials.

### Use of Internal Standards for Nucleotide Site-Specific and Variant-Specific Error Characterization and Control in Targeted NGS Analysis of Cancer Driver Mutations

As shown in FIG. 1, for the targeted driver gene regions spanned in this study, the median technical error VAF measured in IS for corresponding true positive sample variants was 0.014%. This error rate is similar to that reported from other studies that employed targeted NGS on an Illumina platform to assess cancer driver gene hotspot regions.

A key advantage of the presently describe approach is that inclusion of synthetic internal standards with confirmed reference sequence in each library sample preparation enabled qualitative and quantitative characterization of technical error for each variant at each nucleotide site in each library. This approach enabled determination of significance relative to background error for each detected variant in each measurement as is desirable for all diagnostic applications, including those that employ NGS.

Use of synthetic IS as described here for targeted NGS diagnostics is analogous to IS applications that are now standard in liquid and gas chromatography, and mass spectrometry diagnostic application.

As such, use of the low cost, low complexity approach presented here for error control is highly suited to analysis of somatic mutations with VAF>0.05% in driver gene regions. Due to practical limits on size of clinical specimens available for NGS analysis, it is reasonable to consider the specimen-determined lower limit for mutation VAF to be >0.05%.

### Non-Limiting Examples of Applications

In some embodiments, a method for obtaining a numerical index that indicates a biological state comprises providing 2 samples corresponding to each of a first biological state and a second biological state; measuring and/or enumerating an amount of each of 2 nucleic acids in each of the 2 samples; providing the amounts as numerical values that are directly comparable between a number of samples; mathematically computing the numerical values corresponding to each of the first and second biological states; and determining a mathematical computation that discriminates the two biological states. First and second biological states as used herein correspond to two biological states of to be compared, such as two phenotypic states to be distinguished. Non-limiting examples include, e.g., non-disease (normal) tissue vs. disease tissue; a culture showing a therapeutic drug response vs. a culture showing less of the therapeutic drug response; a subject showing an adverse drug response vs. a subject showing a less adverse response; a treated group of subjects vs. a non-treated group of subjects, etc.

A “biological state” as used herein can refer to a phenotypic state, for e.g., a clinically relevant phenotype or other metabolic condition of interest. Biological states can include, e.g., a disease phenotype, a predisposition to a disease state or a non-disease state; a therapeutic drug response or predisposition to such a response, an adverse drug response (e.g. drug toxicity) or a predisposition to such a response, a resistance to a drug, or a predisposition to showing such a resistance, etc. In preferred embodiments, the numerical index obtained can act as a biomarker, e.g., by correlating with a phenotype of interest. In some embodiments, the drug may be and anti-tumor drug. In certain embodiments, the use of the method described herein can provide personalized medicine.

In certain embodiments, the biological state corresponds to a normal expression level of a gene. Where the biological state does not correspond to normal levels, for example falling outside of a desired range, a non-normal, e.g., disease condition may be indicated.

A numerical index that discriminates a particular biological state, e.g., a disease or metabolic condition, can be used as a biomarker for the given condition and/or conditions related thereto.

In some embodiments, one or more of the nucleic acids to be measured are associated with one of the biological states to a greater degree than the other(s). For example, in some embodiments, one or more of the nucleic acids to be evaluated is associated with a first biological state and not with a second biological state.

A nucleic acid may be said to be “associated with” a particular biological state where the nucleic acid is either positively or negatively associated with the biological state. For example, a nucleic acid may be said to be “positively associated” with a first biological state where the nucleic acid occurs in higher amounts in a first biological state compared to a second biological state. As an illustration, genes highly expressed in cancer cells compared to non-cancer cells can be said to be positively associated with cancer. On the other hand, a nucleic acid present in lower amounts in a first biological state compared to a second biological state can be said to be negatively associated with the first biological state.

The nucleic acid to be measured and/or enumerated may correspond to a gene associated with a particular phenotype. The sequence of the nucleic acid may correspond to the transcribed, expressed, and/or regulatory regions of the gene (e.g., a regulatory region of a transcription factor, e.g., a transcription factor for co-regulation).

In some embodiments, expressed amounts of more than 2 genes are measured and used in to provide a numerical index indicative of a biological state. For example, in some cases, expression patterns of multiple genes are used to characterize a given phenotypic state, e.g., a clinically relevant phenotype. In some embodiments, expressed amounts of at least about 5 genes, at least about 10 genes, at least about 20 genes, at least about 50 genes, or at least about 70 genes may be measured and used to provide a numerical index indicative of a biological state. In some embodiments of the instant invention, expressed amounts of less than about 90 genes, less than about 100 genes, less than about 120 genes, less than about 150 genes, or less than about 200 genes may be measured and used to provide a numerical index indicative of a biological state.

Determining which mathematic computation to use to provide a numerical index indicative of a biological state may be achieved by any methods known in the arts, e.g., in the mathematical, statistical, and/or computational arts. In some embodiments, determining the mathematical computation involves a use of software. For example, in some embodiments, a machine learning software can be used.

Mathematically computing numerical values can refer to using any equation, operation, formula and/or rule for interacting numerical values, e.g., a sum, difference, product, quotient, log power and/or other mathematical computation. In some embodiments, a numerical index is calculated by dividing a numerator by a denominator, where the numerator corresponds to an amount of one nucleic acid and the denominator corresponds to an amount the another nucleic acid. In certain embodiments, the numerator corresponds to a gene positively associated with a given biological state and the denominator corresponds to a gene negatively associated with the biological state. In some embodiments, more than one gene positively associated with the biological state being evaluated and more than one gene negatively associated with the biological state being evaluated can be used. For example, in some embodiments, a numerical index can be derived comprising numerical values for the positively associated genes in the numerator and numerical values for an equivalent number of the negatively associated genes in the denominator. In such balanced numerical indices, the reference nucleic acid numerical values cancel out. In some embodiments, balanced numerical values can neutralize effects of variation in the expression of the gene(s) providing the reference nucleic acid(s). In some embodiments, a numerical index is calculated by a series of one or more mathematical functions.

In some embodiments, more than 2 biological states can be compared, e.g., distinguished. For example, in some embodiments, samples may be provided from a range of biological states, e.g., corresponding to different stages of disease progression, e.g., different stages of cancer. Cells in different stages of cancer, for example, include a non-cancerous cell vs. a non-metastasizing cancerous cell vs. a metastasizing cell from a given patient at various times over the disease course. In preferred embodiments, biomarkers can be developed to predict which chemotherapeutic agent can work best for a given type of cancer, e.g., in a particular patient. A non-cancerous cell may include a cell of hematoma and/or scar tissue, as well as morphologically normal parenchyma from non-cancer patients, e.g., non-cancer patients related or not related to a cancer patient. Non-cancerous cells may also include morphologically normal parenchyma from cancer patients, e.g., from a site close to the site of the cancer in the same tissue and/or same organ; from a site further away from the site of the cancer, e.g., in a different tissue and/or organ in the same organ-system, or from a site still further away e.g., in a different organ and/or a different organ-system.

Numerical indices obtained can be provided as a database. Numerical indices and/or databases thereof can find use in diagnoses, e.g. in the development and application of clinical tests.

### Diagnostic Applications

In some embodiments, a method of identifying a biological state is provided. In some embodiments, the method comprises measuring and/or enumerating an amount of each of 2 nucleic acids in a sample, providing the amounts as numerical values; and using the numerical values to provide a numerical index, whereby the numerical index indicates the biological state.

A numerical index that indicates a biological state can be determined as described above in accordance with various embodiments. The sample may be obtained from a specimen, e.g., a specimen collected from a subject to be treated. The subject may be in a clinical setting, including, e.g., a hospital, office of a health care provider, clinic, and/or other health care and/or research facility. Amounts of nucleic acid(s) of interests in the sample can then be measured and/or enumerated.

In certain embodiments, where a given number of genes are to be evaluated, expression data for that given number of genes can be obtained simultaneously. By comparing the expression pattern of certain genes to those in a database, a chemotherapeutic agent that a tumor with that gene expression pattern would most likely respond to can be determined.

In some embodiments, the methods can be used to quantify exogenous normal gene in the presence of mutated endogenous gene. Using primers that span the deleted region, one can selectively amplify and quantitate expression from a transfected normal gene and/or a constitutive abnormal gene.

In some embodiments, methods described herein can be used to determine normal expression levels, e.g., providing numerical values corresponding to normal gene transcript expression levels. Such embodiments may be used to indicate a normal biological state, at least with respect to expression of the evaluated gene.

Normal expression levels can refer to the expression level of a transcript under conditions not normally associated with a disease, trauma, and/or other cellular insult. In some embodiments, normal expression levels may be provided as a number, or preferably as a range of numerical values corresponding to a range of normal expression of a particular gene, e.g., within +/−a percentage for experimental error. Comparison of a numerical value obtained for a given nucleic acid in a sample, e.g., a nucleic acid corresponding to a particular gene, can be compared to established-normal numerical values, e.g., by comparison to data in a database provided herein. As numerical values can indicate numbers of molecules of the nucleic acid in the sample, this comparison can indicate whether the gene is being expressed within normal levels or not.

In some embodiments, the method can be used for identifying a biological state comprising assessing an amount a nucleic acid in a first sample, and providing said amount as a numerical value wherein said numerical value is directly comparable between a number of other samples. In some embodiments, the numerical value is potentially directly comparable to an unlimited number of other samples. Samples may be evaluated at different times, e.g., on different days; in the same or different experiments in the same laboratory; and/or in different experiments in different laboratories.

### Therapeutic Applications

Some embodiments provide a method of improving drug development. For example, use of a standardized mixture of internal standards, a database of numerical values and/or a database of numerical indices may be used to improve drug development.

In some embodiments, modulation of gene expression is measured and/or enumerated at one or more of these stages, e.g., to determine effect a candidate drug. For example, a candidate drug (e.g., identified at a given stage) can be administered to a biological entity. The biological entity can be any entity capable of harboring a nucleic acid, as described above, and can be selected appropriately based on the stage of drug development. For example, at the lead identification stage, the biological entity may be an in vitro culture. At the stage of a clinical trial, the biological entity can be a human patient.

The effect of the candidate drug on gene expression may then be evaluated, e.g., using various embodiments of the instant invention. For example, a nucleic acid sample may be collected from the biological entity and amounts of nucleic acids of interest can be measured and/or enumerated. For example, amounts can be provided as numerical value and/or numerical indices. An amount then may be compared to another amount of that nucleic acid at a different stage of drug development; and/or to a numerical values and/or indices in a database. This comparison can provide information for altering the drug development process in one or more ways.

Altering a step of drug development may refer to making one or more changes in the process of developing a drug, preferably so as to reduce the time and/or expense for drug development. For example, altering may comprise stratifying a clinical trial. Stratification of a clinical trial can refer to, e.g., segmenting a patient population within a clinical trial and/or determining whether or not a particular individual may enter into the clinical trial and/or continue to a subsequent phase of the clinical trial. For example, patients may be segmented based on one or more features of their genetic makeup determined using various embodiments of the instant invention. For example, consider a numerical value obtained at a pre-clinical stage, e.g., from an in vitro culture that is found to correspond to a lack of a response to a candidate drug. At the clinical trial stage, subjects showing the same or similar numerical value can be exempted from participation in the trial. The drug development process has accordingly be altered, saving time, and costs.

### Kits

The internal amplification control (IAC)/competitive internal standards (IS) described herein may be assembled and provided in the form of kits. In some embodiments, the kit provides the IAC and reagents necessary to perform a PCR, including Multiplex-PCR and next-generation sequencing (NGS). The IAC may be provided in a single, concentrated form where the concentration is known, or serially diluted in solution to at least one of several known working concentrations.

The kits may include IS of 150 identified endogenous targets, as described herein, or IS of 28 ERCC (External RNA Control Consortium) targets, as described herein, or both. These IS may be provided in solution allowing the IS to remain stable for up to several

years.

The kits may also provide primers designed specifically to amplify the IS of 150 endogenous targets, the IS of 28 ERCC targets, and their corresponding native targets.

The kits may also provide one or more containers filled with one or more necessary PCR reagents, including but not limited to dNTPs, reaction buffer, Taq polymerase, and RNAse-free water. Optionally associated with such container(s) is a notice in the form prescribed by a governmental agency regulating the manufacture, use or sale of IAC and associated reagents, which notice reflects approval by the agency of manufacture, use or sale for research use.

The kits may include appropriate instructions for preparing, executing, and analyzing PCR, including Multiplex-PCR and NGS, using the IS included in the kit. The instructions may be in any suitable format, including, but not limited to, printed matter, videotape, computer readable disk, or optical disc.

All publications, including patents and non-patent literature, referred to in this specification are expressly incorporated by reference herein. Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

What is claimed is:

1. A lung cancer risk test kit, comprising:
reagents for measurement of multiple low variant allele frequency (VAF) mutants in a set of lung cancer driver genes; wherein reagents include:
a) polymerase chain reaction (PCR) primers for each target gene,
b) synthetic internal standard for each target gene, and
c) reagents to prepare PCR products as a library for next generation sequencing; and, instructions therefor.

2. The kit of claim 1, wherein the set of lung cancer driver genes comprise one or more of: TP53, PIK3CA, BRAF, KRAS, NRAS, NOTCHI, EGFR, and ERBB2.

3. The kit of claim 1, wherein the set of lung cancer driver genes comprise one or more of: CDKN1A, E2F1, ERCC1, ERCC4, ERCC5, GPX1, GSTP1, KEAP1, RB1, TP63, and XRCC1.

4. The kit of claim 1, wherein the kit provides reagents and instructions necessary for measurement of multiple VAF mutants.

5. The kit of claim 1, wherein the kit provides reagents and instructions necessary for conducting tests in multiple patient specimens.

6. The kit of claim 1, wherein the kit further provides one or more sets of instructions to use the kit to:
facilitate approval by the Food and Drug Administration (FDA) and other regulatory agencies of lung cancer risk testing in regional laboratories;
facilitate approval by FDA and other regulatory agencies of testing for measurement of mutations in cancer cells that will then guide targeted therapy of the cancer in regional laboratories; or
facilitate approval by FDA and other regulatory agencies of testing for measurement without unique molecular indices (UMI) of very low VAF (as low as 0.01%) mutations in cancer cells that will then guide targeted therapy of the cancer in kit or method form in regional laboratories.

7. The kit of claim 1, wherein the kit further provides one or more sets of instructions to use the kit to:
enable measurement of lung cancer risk in non-invasively obtained specimens, including exhaled breath condensate, bronchial brush and nasal brush specimens;
enable measurement of very low VAF mutations in airway epithelial cells;
measure mutations in cancer cells that will then guide targeted therapy of the cancer; and, measure mutations in a set of genes in normal airway cells to determine risk for cancer.
